(19) United States
(12) Patent Application Publication
(10) Pub. No.: US 2018/0253590 A1
(43) Pub. Date: Sep. 6, 2018

(54) SYSTEMS, METHODS, AND APPARATUSES FOR DIGITAL HISTOPATHOLOGICAL IMAGING FOR PRESCREENED DETECTION OF CANCER AND OTHER ABNORMALITIES

(71) Applicant: Inspirata, Inc., Tampa, FL (US)

(72) Inventors: Mark C. Lloyd, Tampa, FL (US); James Monaco, Odessa, FL (US); Nishant Verma, Tampa, FL (US); David Scott Harding, Tampa, FL (US); Maykel Orozco Monteagudo, Tampa, FL (US); Kirk William Gossage, Wesley Chapel, FL (US); Janani Sivasankar Babu, Tampa, FL (US)

(21) Appl. No.: 15/559,708

(22) PCT Filed: Mar. 21, 2016
(86) PCT No.: PCT/US16/23421

§ 371 Date: Sep. 19, 2017

Related U.S. Application Data
(60) Provisional application No. 62/136,051, filed on Mar. 20, 2015.

(51) Int. Cl. G06K 9/00; G01N 33/483; G06K 9/46; G06T 7/00

(52) U.S. Cl. CPC G06K 9/00147; G01N 33/4833; G06T 2207/30024; G06T 7/0012; G06K 9/4652

(57) ABSTRACT

Systems, methods, and apparatuses for analyzing histopathology images to determine the presence of certain predetermined abnormalities. The system processes histopathology images to identify/highlight regions of interest (e.g., a region that may comprise parts of a tumor, cancerous cells, or other predetermined abnormality) for subsequent review by a pathologist or other trained professional. For example, the system may process histopathology images of H&E-stained lymph node tissue to identify potentially cancerous cells within the histopathology images.

### CROSS REFERENCE TO RELATED APPLICATIONS

This application claims priority to, the benefit under 35 U.S.C. § 119 of, and incorporates by reference herein in its entirety U.S. Provisional Patent Application No. 62/136,051, filed Mar. 20, 2015, and entitled “Systems, Methods, and Apparatuses for Digital Whole Slide Imaging for Prescreened Detection of Cancer and Other Abnormalities.”

### TECHNICAL FIELD

The present systems, methods, and apparatuses relate generally to digital histopathological imaging and, more particularly, to analyzing histopathology images to determine the presence of certain predetermined abnormalities.

### BACKGROUND

Pathologists review histopathology images to determine whether any abnormalities are present in the tissue within the image. For example, a pathologist may review histopathology images of tissue collected during a visit to a dermatologist to determine whether a mole is a carcinoma. Or, a pathologist may review tissue collected from a patient during surgery (while the patient is still under anesthesia) to determine whether a tumor has been completely removed. Anytime a pathologist reviews a histopathology image, the pathologist must review the entire image to determine whether a particular abnormality is present, which can be a slow and tedious process. In some cases, a resident physician is assigned the task of initially reviewing histopathology images to identify potential abnormalities for review by the pathologist, which also can further delay the histopathology review process.

Therefore, there is a long-felt but unresolved need for a system, method, or apparatus that quickly and efficiently analyzes histopathology images to determine the presence of certain predetermined abnormalities.

### BRIEF SUMMARY OF THE DISCLOSURE

Briefly described, and according to one embodiment, aspects of the present disclosure generally relate to systems, methods, and apparatuses for analyzing histopathology images to determine the presence of certain predetermined abnormalities.

Histopathology images generally comprise digitized versions of microscopic views of tissue slides that may contain pieces of tissue from various human organs or abnormal masses within the body (e.g., lymph node, tumor, skin, etc.). These histopathology images may be collected for review by a trained professional (e.g., pathologist) to diagnose a particular disease, determine whether a tumor is malignant or benign, determine whether a surgeon has completely excised a tumor, etc. To expedite the review process (because pathologists view hundreds of histopathology images daily and often make only a binary decision regarding a particular histopathology image: yes, the image contains cancerous cells or, no, the image does not contain cancerous cells), the tissue analysis system described in the present disclosure processes histopathology images to identify/highlight regions of interest (e.g., a region that may comprise parts of a tumor, cancerous cells, or other predetermined abnormality), typically for subsequent review by a pathologist or other trained professional. Generally, by identifying/highlighting regions of interest within the histopathology images, the pathologist's review time of a particular histopathology image is reduced because the review need only cover the regions of interest and not the entire histopathology image. In some embodiments, professionals need not review the results of the processing because the tissue analysis system automatically identifies abnormalities within the histopathology images.

The tissue analysis system may, in various embodiments, process multiple histopathology images either concurrently or simultaneously to identify regions of interest in each of the histopathology images. In various embodiments, the identification of regions of interest within a histopathology image comprises the following processes: tissue identification, artifact removal, low-resolution analysis, and high-resolution analysis. In one embodiment, tissue identification is the process by which the present tissue analysis system identifies tissue regions (and, in one embodiment, a particular type of tissue) within the histopathology image (e.g., separating the tissue regions from the blank background regions). Generally, tissue identification increases the accuracy and efficiency of the tissue analysis system. Artifact removal, in one embodiment, is the process by which the tissue analysis system removes artifacts (e.g., blurry regions, fingerprints, foreign objects such as dust or hair, etc.) that may have accidentally been included on the tissue slide from the histopathology image, also increasing the accuracy and efficiency of the tissue analysis system. In one embodiment, low-resolution analysis is the process by which the tissue analysis system identifies potential regions of interest, with an emphasis on speed and/or low-resource processing (not necessarily accuracy), for subsequent confirmation as regions of interest based on certain predefined features within the identified tissue (e.g., cellular structures, nuclei patterns, etc.). High-resolution analysis, in one embodiment, is the process by which the tissue analysis system confirms whether a particular potential region of interest should be considered a region of interest, based on predefined nuclei patterns, for subsequent analysis by a professional. In various embodiments, the identified regions of interest (and other parts of the process as disclosed herein) are flagged and stored with the histopathology image as a layer(s) on top of the histopathology image that may be viewed (or removed from view) by the professional.

For example, to determine whether a patient has cancer, the tissue analysis system may process the histopathology image(s) of lymph node tissue to identify regions of interest that may contain cancerous cells. Accordingly, the histopathology image(s) undergo the tissue identification process to identify the lymph node tissue within the histopathology image(s) and confirm that the tissue is lymph node tissue and not some other tissue (e.g., adipose tissue, etc.). Similarly, the histopathology image(s) undergo the artifact removal process to remove any artifacts contained within the histopathology image(s). The histopathology image(s) undergo the low-resolution analysis process to quickly identify potential regions of interest for further analysis during the high-resolution analysis process, and the high-resolution analysis process, during which the tissue analysis system identifies/flags, for subsequent review by a pathologist, regions of interest that may contain cancerous cells. Thus, the pathologist may quickly review the histopathology image(s) of the lymph node tissue to determine whether a patient has cancer.

In one embodiment, a method for processing images of cells to identify cellular nuclei within the cells for use in connection with identifying a possible abnormality with respect to the cells, comprising the steps of: receiving an image of one or more cells, each cell having a cellular nucleus, wherein the image of the one or more cells comprises a plurality of pixels of varying brightness; applying a sampling matrix to each of the plurality of pixels of the image of the one or more cells, wherein the sampling matrix determines one or more first and second derivatives with respect to the brightness of a particular pixel to which the sampling matrix was applied; determining a consistency for each of the one or more first and second derivatives; and selecting, based on the determined consistency for each of the one or more first and second derivatives, one or more edges of a cellular nucleus within the image of the one or more cells, wherein the selected one or more edges of the cellular nucleus help define the shape of the cellular nucleus.

In one embodiment, a system for processing images of cells to identify cellular nuclei within the cells for use in connection with identifying a possible abnormality with respect to the cells, comprising: one or more electronic computing devices; and a processor operatively connected to the one or more electronic computing devices, wherein the processor is operative to: receive an image of one or more cells from the one or more electronic computing devices, each cell having a cellular nucleus, wherein the image of the one or more cells comprises a plurality of pixels of varying brightness; apply a sampling matrix to each of the plurality of pixels of the image of the one or more cells, wherein the sampling matrix determines one or more first and second derivatives with respect to the brightness of a particular pixel to which the sampling matrix was applied; determine a consistency for each of the one or more first and second derivatives; and select, based on the determined consistency for each of the one or more first and second derivatives, one or more edges of a cellular nucleus within the image of the one or more cells, wherein the selected one or more edges of the cellular nucleus help define the shape of the cellular nucleus.

In one embodiment, a method for processing images of cells to identify cellular nuclei within the cells and to determine nuclei shapes within the cells for use in connection with identifying a possible abnormality with respect to the cells, comprising the steps of: receiving an image of one or more cells, each cell having a cellular nucleus, wherein the image of the one or more cells comprises a plurality of pixels of varying brightness and shape data regarding at least one particular nucleus within the image of the one or more cells; selecting, based on the shape data, an initial pixel within the at least one particular nucleus from which to determine the shape of the at least one particular nucleus; adding additional pixels to the initial pixel, based on one or more predefined rules, until the number of pixels within the at least one particular nucleus exceeds a predetermined threshold value; and determining, based on the additional pixels, the shape of the at least one particular nucleus.

In one embodiment, a system for processing images of cells to identify cellular nuclei within the cells and to determine nuclei shapes within the cells for use in connection with identifying a possible abnormality with respect to the cells, comprising: one or more electronic computing devices; and a processor operatively connected to the one or more electronic computing devices, wherein the processor is operative to: receive, from the one or more electronic computing devices, an image of one or more cells, each cell having a cellular nucleus, wherein the image of the one or more cells comprises a plurality of pixels of varying brightness and shape data regarding at least one particular nucleus within the image of the one or more cells; select, based on the shape data, an initial pixel within the at least one particular nucleus from which to determine the shape of the at least one particular nucleus; add additional pixels to the initial pixel, based on one or more predefined rules, until the number of pixels within the at least one particular nucleus exceeds a predetermined threshold value; and determine, based on the additional pixels, the shape of the at least one particular nucleus.

In one embodiment, a method for processing images of cells comprising cellular nuclei to determine the presence of an abnormality within the cells, comprising the steps of: receiving an image of one or more cells, each cell having a cellular nucleus, wherein the image of the one or more cells comprises a plurality of pixels of varying brightness; identifying, based on the brightness of the plurality of pixels, one or more edges corresponding to a particular cellular nucleus; defining, based on the identified one or more edges and the plurality of pixels, a shape of the particular cellular nucleus; and comparing the shape of the particular cellular nucleus to one or more predefined rules to determine whether the shape of the particular cellular nucleus indicates the presence of the abnormality in the one or more cells.

In one embodiment, a system for processing images of cells comprising cellular nuclei to determine the presence of an abnormality within the cells, comprising: one or more electronic computing devices; and a processor operatively connected to the one or more electronic computing devices, wherein the processor is operative to: receive, from the one or more electronic computing devices, an image of one or more cells, each cell having a cellular nucleus, wherein the image of the one or more cells comprises a plurality of pixels of varying brightness; identify, based on the brightness of the plurality of pixels, one or more edges corresponding to a particular cellular nucleus; define, based on the identified one or more edges and the plurality of pixels, a shape of the particular cellular nucleus; and compare the shape of the particular cellular nucleus to one or more predefined rules to determine whether the shape of the particular cellular nucleus indicates the presence of the abnormality in the one or more cells.

According to one aspect of the present disclosure, the method, wherein the sampling matrix comprises an arc-shaped filter. Furthermore, the method, wherein determining the consistency for each of the one or more first and second derivatives further comprises the steps of: determining an arithmetic mean for each of the one or more first and second derivatives; and determining a standard deviation for each of the one or more first and second derivatives. Moreover, the method, wherein the one or more first and second derivatives comprise one or more arc lengths. Further, the method, wherein the consistency for each of the one or more first and second derivatives is determined for each of the one or more arc lengths. Additionally, the method, wherein selecting the one or more edges of the cellular nucleus within the image of the one or more cells further comprises the steps of: converting the determined consistency for each of the one or more first and second derivatives into a normalized signal-to-noise ratio value; and selecting the one or more edges of the cellular nucleus within the image of the one or more cells corresponding to the determined consistency for each of the one or more first and second derivatives with the maximum normalized signal-to-noise ratio value. Also, the method, wherein the image of the one or more cells comprises a preprocessed image of the one or more cells.

According to one aspect of the present disclosure, the system, wherein the sampling matrix comprises an arc-shaped filter. Furthermore, the system, wherein to determine the consistency for each of the one or more first and second derivatives, the processor is further operative to: determine an arithmetic mean for each of the one or more first and second derivatives; and determine a standard deviation for each of the one or more first and second derivatives. Moreover, the system, wherein the one or more first and second derivatives comprise one or more arc lengths. Further, the system, wherein the consistency for each of the one or more first and second derivatives is determined for each of the one or more arc lengths. Additionally, the system, wherein to select the one or more edges of the cellular nucleus within the image of the one or more cells, the processor is further operative to: convert the determined consistency for each of the one or more first and second derivatives into a normalized signal-to-noise ratio value; and select the one or more edges of the cellular nucleus within the image of the one or more cells corresponding to the determined consistency for each of the one or more first and second derivatives with the maximum normalized signal-to-noise ratio value. Also, the system, wherein the processor is further operative to, prior to applying the sampling matrix to each of the plurality of pixels, preprocess the image of the one or more cells. In addition, the system, wherein the one or more electronic computing devices further comprise one or more slide scanners.

According to one aspect of the present disclosure, the method, wherein the shape data comprises data corresponding to one or more edges of the at least one particular nucleus and data regarding one or more initial pixels within the at least particular one nucleus. Furthermore, the method, wherein the one or more predefined rules define, based on one or more multivariate normal distribution intensities of the brightness of the additional pixels, the additional pixels most likely to be within the at least particular one nucleus. Moreover, the method, wherein the one or more multivariate normal distribution intensities are determined based on the brightness of the additional pixels and the shape data. Further, the method, wherein the shape data comprises the predetermined threshold value. Additionally, the method, further comprising the step of determining, after each additional pixel is added to the initial pixel, a fitness of a current shape of the at least one particular nucleus, wherein the fitness corresponds to the accuracy of the current shape of the at least one particular nucleus. Also, the method, wherein the shape of the at least one particular nucleus is determined based on the fitness determined after each additional pixel was added to the initial pixel. In addition, the method, wherein the image of the one or more cells comprises a preprocessed image of the one or more cells.

According to one aspect of the present disclosure, the system, wherein the shape data comprises data corresponding to one or more edges of the at least one particular nucleus and data regarding one or more initial pixels within the at least particular one nucleus. Furthermore, the system, wherein the one or more predefined rules define, based on one or more multivariate normal distribution intensities of the brightness of the additional pixels, the additional pixels most likely to be within the at least particular one nucleus. Moreover, the system, wherein the one or more multivariate normal distribution intensities are determined based on the brightness of the additional pixels and the shape data. Further, the system, wherein the shape data comprises the predetermined threshold value. Additionally, the system, further comprising the step of determining, after each additional pixel is added to the initial pixel, a fitness of a current shape of the at least one particular nucleus, wherein the fitness corresponds to the accuracy of the current shape of the at least one particular nucleus. Likewise, the system, wherein the shape of the at least one particular nucleus is determined based on the fitness determined after each additional pixel was added to the initial pixel. Also, the system, wherein the processor is further operative to, prior to selecting the initial pixel, preprocess the image of the one or more cells. In addition, the system, wherein the one or more electronic computing devices further comprise one or more slide scanners.

According to one aspect of the present disclosure, the method, wherein identifying the one or more edges further comprises the steps of: applying a sampling matrix to each of the plurality of pixels of the image of the one or more cells, wherein the sampling matrix determines one or more first and second derivatives with respect to the brightness of a particular pixel to which the sampling matrix was applied; determining a consistency for each of the one or more first and second derivatives; and selecting, based on the determined consistency for each of the one or more first and second derivatives, one or more edges of a cellular nucleus within the image of the one or more cells, wherein the selected one or more edges of the cellular nucleus help define the shape of the cellular nucleus. Furthermore, the method, wherein defining the shape of the particular cellular nucleus further comprises the steps of: selecting, based on the identified one or more edges and the plurality of pixels, an initial pixel within the one particular nucleus from which to determine the shape of the particular nucleus; adding additional pixels to the initial pixel, based on one or more predefined rules, until the number of pixels within the particular nucleus exceeds a predetermined threshold value; and determining, based on the additional pixels, the shape of the particular nucleus. Moreover, the method, wherein the one or more predefined rules comprise data regarding the characteristics of cellular nuclei comprising the particular abnormality. Further, the method, wherein the characteristics of nuclei are selected from the group comprising: a shape of the cellular nuclei, a size of the cellular nuclei, a spatial relationship between the cellular nuclei, and a number of the cellular nuclei within a region of predetermined size.

According to one aspect of the present disclosure, the method, further comprising the step of, prior to identifying the one or more edges, preprocessing the image of the one or more cells. Additionally, the method, wherein preprocessing the image of the one or more cells further comprises the step of identifying tissue comprising the one or more cells within the image of the one or more cells. Also, the method, wherein preprocessing the image of the one or more cells further comprises the steps of identifying one or more artifacts within the image of the one or more cells and removing the identified one or more artifacts from the image of the one or more cells. Furthermore, the method, wherein preprocessing the image of the one or more cells further comprises the step of converting the image of the one or more cells to a particular color space. Moreover, the method, wherein preprocessing the image of the one or more cells further comprises the step of extracting one or more particular color channels from the image of the one or more cells. Further, the method, wherein preprocessing the image of the one or more cells further comprises the step of selecting a particular image size for the image of the one or more cells. Additionally, the method, wherein preprocessing the image of the one or more cells further comprises the step of identifying one or more texture features within the image of the one or more cells. Also, the method, wherein preprocessing the image of the one or more cells further comprises the step of dividing the plurality of pixels into one or more groups of predetermined size.

According to one aspect of the present disclosure, the system, wherein to identify the one or more edges, the processor is further operative to: apply a sampling matrix to each of the plurality of pixels of the image of the one or more cells, wherein the sampling matrix determines one or more first and second derivatives with respect to the brightness of a particular pixel to which the sampling matrix was applied; determine a consistency for each of the one or more first and second derivatives; and select, based on the determined consistency for each of the one or more first and second derivatives, one or more edges of a cellular nucleus within the image of the one or more cells, wherein the selected one or more edges of the cellular nucleus help define the shape of the cellular nucleus. Furthermore, the system, wherein to define the shape of the particular cellular nucleus, the process is further operative to: select, based on the identified one or more edges and the plurality of pixels, an initial pixel within the one particular nucleus from which to determine the shape of the particular nucleus; add additional pixels to the initial pixel, based on one or more predefined rules, until the number of pixels within the particular nucleus exceeds a predetermined threshold value; and determine, based on the additional pixels, the shape of the particular nucleus. Moreover, the system, wherein the one or more predefined rules comprise data regarding the characteristics of cellular nuclei comprising the particular abnormality. Further, the system, wherein the characteristics of nuclei are selected from the group comprising: a shape of the cellular nuclei, a size of the cellular nuclei, a spatial relationship between the cellular nuclei, and a number of the cellular nuclei within a region of predetermined size. Additionally, the system, wherein the one or more electronic computing devices further comprise one or more slide scanners.

According to one aspect of the present disclosure, the system, wherein the processor, prior to identifying the one or more edges, is further operative to preprocess the image of the one or more cells. Also, the system, wherein to preprocess the image of the one or more cells, the process is further operative to identify tissue comprising the one or more cells within the image of the one or more cells. Furthermore, the system, wherein to preprocess the image of the one or more cells, the process is further operative to identify one or more artifacts within the image of the one or more cells and remove the identified one or more artifacts from the image of the one or more cells. Moreover, the system, wherein to preprocess the image of the one or more cells, the process is further operative to convert the image of the one or more cells to a particular color space. Further, the system, wherein to preprocess the image of the one or more cells, the process is further operative to extract one or more particular color channels from the image of the one or more cells. Additionally, the system, wherein to preprocess the image of the one or more cells, the process is further operative to select a particular image size for the image of the one or more cells. Also, the system, wherein to preprocess the image of the one or more cells, the process is further operative to identify one or more texture features within the image of the one or more cells. Additionally, the system, wherein to preprocess the image of the one or more cells, the process is further operative to divide the plurality of pixels into one or more groups of predetermined size.

These and other aspects, features, and benefits of the claimed invention(s) will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 illustrates an exemplary, high-level overview of one embodiment of the disclosed system.

FIG. 2 illustrates an exemplary architecture of one embodiment of the disclosed system.

FIG. 3 is a flowchart showing an exemplary process overview, according to one embodiment of the present disclosure.

FIG. 4 is a flowchart showing an exemplary tissue identification process, according to one embodiment of the present disclosure.

FIG. 5 is a flowchart showing an exemplary artifact removal process, according to one embodiment of the present disclosure.

FIG. 6 is a flowchart showing an exemplary low-resolution analysis process, according to one embodiment of the present disclosure.

FIG. 7 (consisting of FIGS. 7A, 7B, 7C, 7D, and 7E) is a flowchart showing an exemplary high-resolution analysis process, according to one embodiment of the present disclosure.

FIG. 8 (consisting of FIGS. 8A, 8B, 8C, 8D, 8E, 8F, 8G, and 8H) illustrates exemplary histopathology images, according to one embodiment of the present disclosure.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

### Overview

Aspects of the present disclosure generally relate to systems, methods, and apparatuses for analyzing histopathology images to determine the presence of certain predetermined abnormalities.

The tissue analysis system may, in various embodiments, process multiple histopathology images either concurrently or simultaneously to identify regions of interest in each of the histopathology images. In various embodiments, the identification of regions of interest within a histopathology image comprises the following processes: tissue identification, artifact removal, low-resolution analysis, and high-resolution analysis. In one embodiment, tissue identification is the process by which the present tissue analysis system identifies tissue regions (and, in one embodiment, a particular type of tissue) within the histopathology image (e.g., separating the tissue regions from the blank background regions). Generally, tissue identification increases the accuracy and efficiency of the tissue analysis system. Artifact removal, in one embodiment, is the process by which the tissue analysis system removes artifacts (e.g., blurry regions, fingerprints, foreign objects such as dust or hair, etc.) that may have accidentally been included on the tissue slide from the histopathology image, also increasing the accuracy and efficiency of the tissue analysis system. In one embodiment, low-resolution analysis is the process by which the tissue analysis system identifies potential regions of interest, with an emphasis on speed and/or low-resource processing (not necessarily accuracy), for subsequent confirmation as regions of interest based on certain predefined features within the identified tissue (e.g., cellular structures, nuclei patterns, etc.). High-resolution analysis, in one embodiment, is the process by which the tissue analysis system confirms whether a particular potential region of interest should be considered a region of interest, based on predefined nuclei patterns, for subsequent analysis by a professional. In various embodiments, the identified regions of interest (and other parts of the process as disclosed herein) are flagged and stored with the histopathology image as a layer(s) on top of the histopathology image that may be viewed (or removed from view) by the professional.

For example, to determine whether a patient has cancer, the tissue analysis system may process the histopathology image(s) of lymph node tissue to identify regions of interest that may contain cancerous cells. Accordingly, the histopathology image(s) undergo the tissue identification process to identify the lymph node tissue within the histopathology image(s) and confirm that the tissue is lymph node tissue and not some other tissue (e.g., adipose tissue, etc.). Similarly, the histopathology image(s) undergo the artifact removal process to remove any artifacts contained within the histopathology image(s). The histopathology image(s) undergo the low-resolution analysis process to quickly identify potential regions of interest for further analysis during the high-resolution analysis process, and the high-resolution analysis process, during which the tissue analysis system identifies/flags, for subsequent review by a pathologist, regions of interest that may contain cancerous cells. Thus, the pathologist may quickly review the histopathology image(s) of the lymph node tissue to determine whether a patient has cancer.

### Exemplary Embodiments

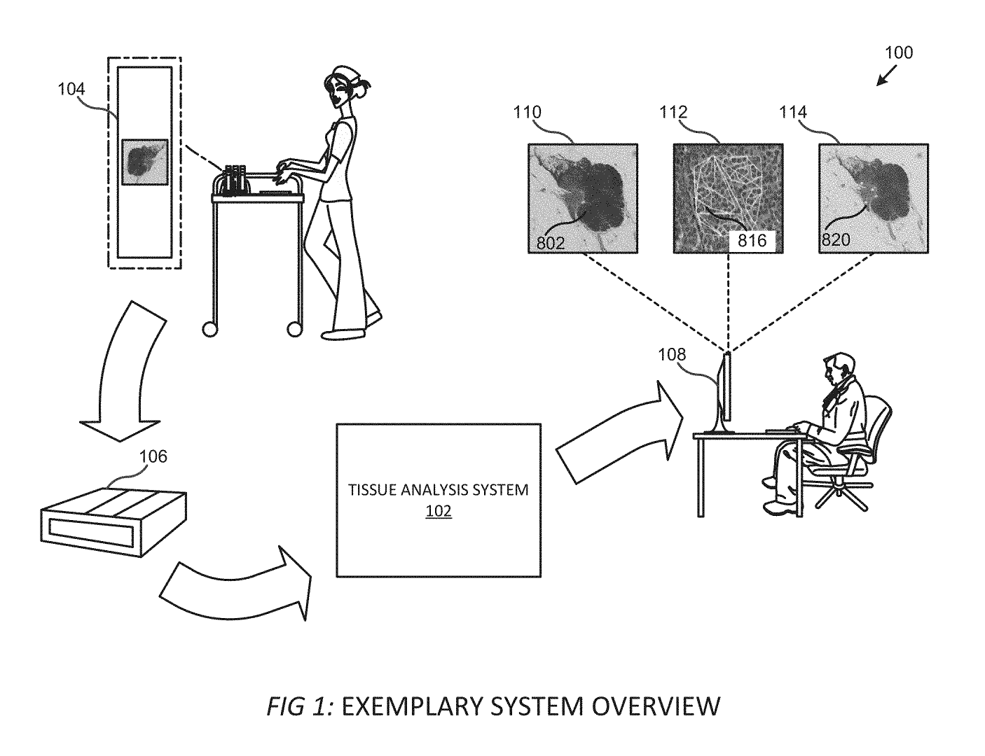

Referring now to the figures, for the purposes of example and explanation of the fundamental processes and components of the disclosed systems, methods, and apparatuses, reference is made to FIG. 1, which illustrates an exemplary, high-level overview 100 of one embodiment of a tissue analysis system 102. As will be understood and appreciated, the exemplary, high-level overview 100 shown in FIG. 1 represents merely one approach or embodiment of the present system, and other aspects are used according to various embodiments of the present system.

Generally, the tissue analysis system 102 processes histopathology images to identify regions of interest of the images, often for subsequent analysis by a trained professional (e.g., pathologist). In various embodiments, histopathology images are digitized versions of microscopic views of tissue slides 104 (e.g., whole slide images, etc.) that may contain pieces of tissue from various human organs (e.g., lymph node, tumor, skin, etc.). These histopathology images may be collected to diagnosis a particular disease, determine whether a tumor is malignant or benign, determine whether a surgeon has completely excised a tumor, etc. Accordingly, a pathologist may view hundreds of these histopathology images every day to make those determinations. To expedite the review process (because pathologists often only making a binary decision regarding a particular histopathology image: yes, the image contains cancerous cells or, no, the image does not contain cancerous cells), the tissue analysis system 102 process histopathology images to identify/highlight regions of interest (e.g., a region that may contain parts of a tumor, cancerous cells, etc.). In one embodiment, a professional (e.g., pathologist) reviews the results of the processing to confirm the presence of the abnormality. In another embodiment, professionals need not review the results of the processing because the tissue analysis system 102 automatically identifies abnormalities within the histopathology images.

In various embodiments, the tissue slides 104 may comprise any slide capable of receiving tissue samples (e.g., glass, plastic, etc.). Thus, the tissue slides 104 usually contain a small, thin piece of tissue that has been excised from a patient for a specific purpose (e.g., diagnose cancer, confirm removal of tumor, etc.). In various embodiments, the tissue is stained to increase the visibility of certain features of the tissue (e.g., using a hematoxylin and eosin/H&E stain, etc.). Traditionally, tissue slides 104 were viewed in painstaking fashion via a microscope. More recently, the tissues slides 104 are scanned by a slide scanner 106 to generate histopathology images, so that pathologists need not use microscopes to view the tissue. Regardless, the pathologist must still review the entirety of the histopathology images to detect abnormalities. Generally, the tissue slides 104 may be loaded automatically into the slide scanner 106 at a rapid rate or may be fed individually into the slide scanner 106 by a technician or other professional. Accordingly, the slide scanner 106 generates a histopathology image that may comprise a detailed, microscopic view of the tissue slide 104 (e.g., an image with dimensions of 80,000×60,000 pixels, wherein 1 pixel is approximately 0.25 microns).

For example, to confirm whether a patient has cancer, a biopsy of the patient's lymph nodes may be performed so that a pathologist may determine whether cancerous cells are present within the patient's lymph nodes. Continuing with this example, the tissue retrieved by that biopsy may be placed on a tissue slide 104, which is fed into the slide scanner 106 to convert the tissue slide into a histopathology image that comprises a detailed view of the lymph node tissue sample. In another example, to confirm whether a surgeon has completely removed a tumor during surgery, a biopsy of the exterior portions of the removed tumor may be taken so that a pathologist may determine whether the tissue comprises the exterior of the tumor (thus, signifying that the tumor has been completely removed) or an interior portion of the tumor (thus, indicating that the surgeon must remove more of the tumor). The tissue retrieved by that biopsy may be placed on a tissue slide 104, which is fed into the slide scanner 106 to convert the tissue slide into a histopathology image that comprises a detailed view of the tumor tissue sample.

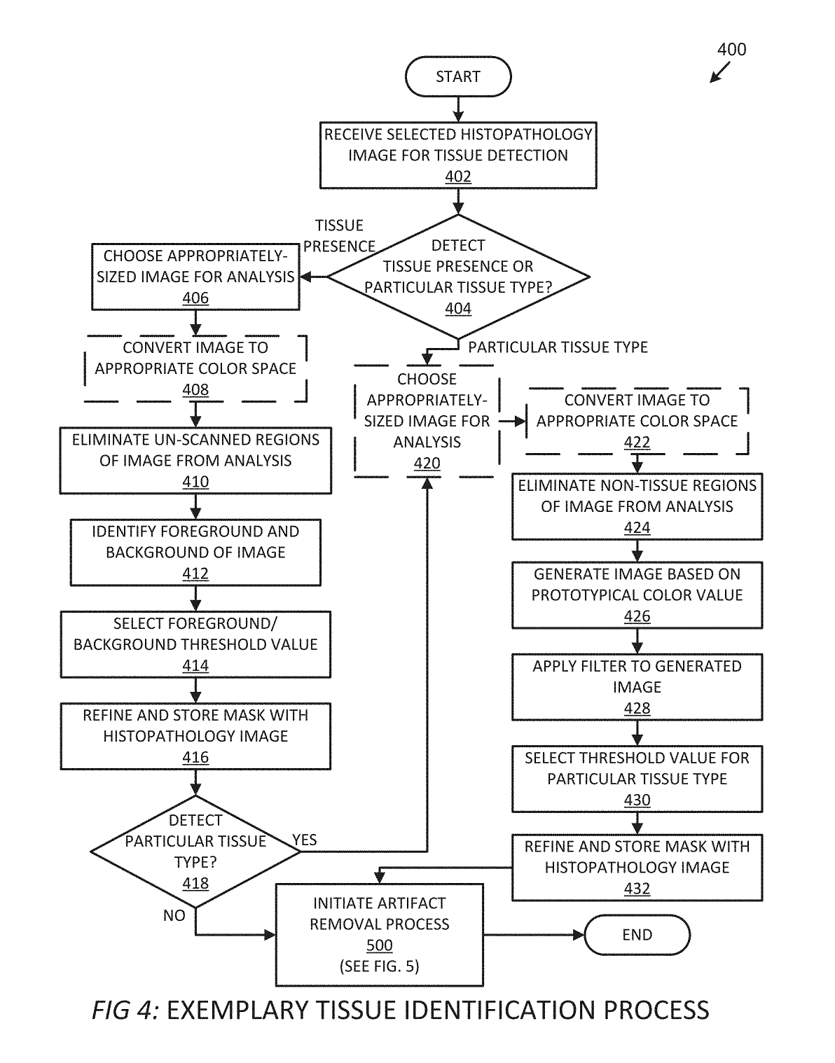

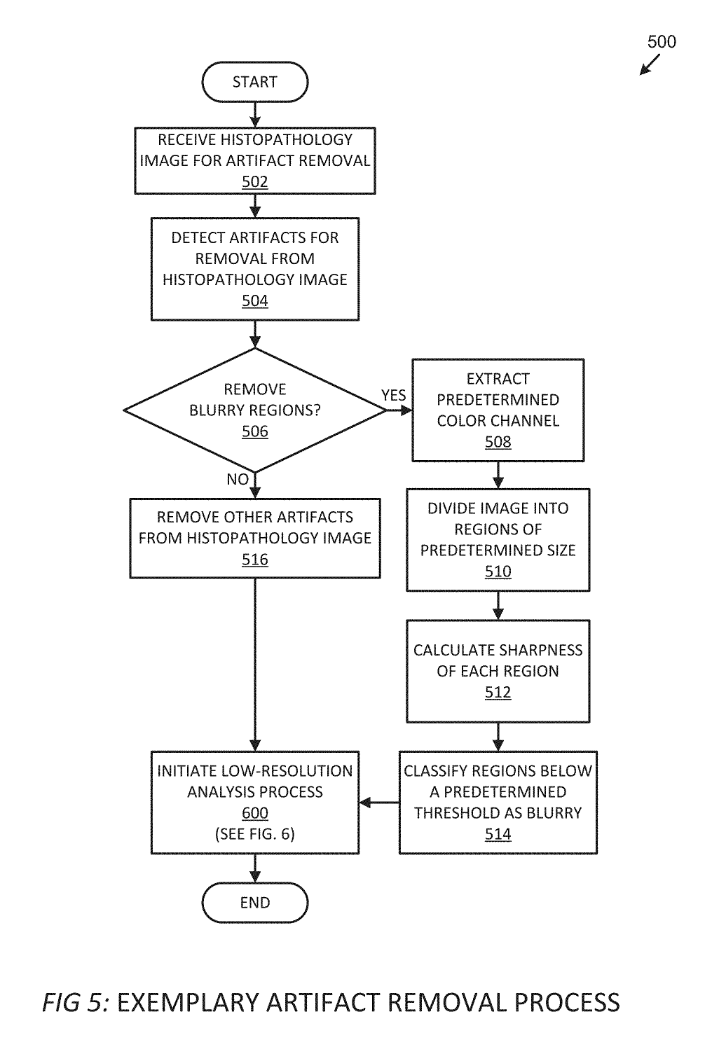

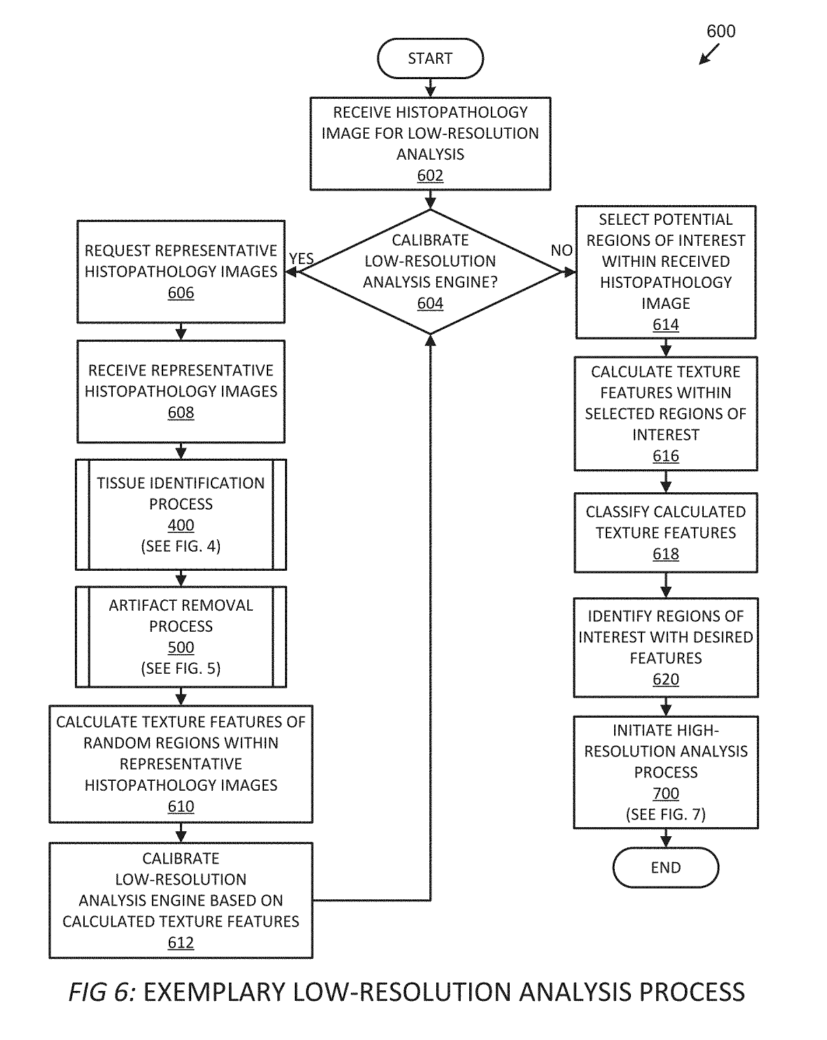

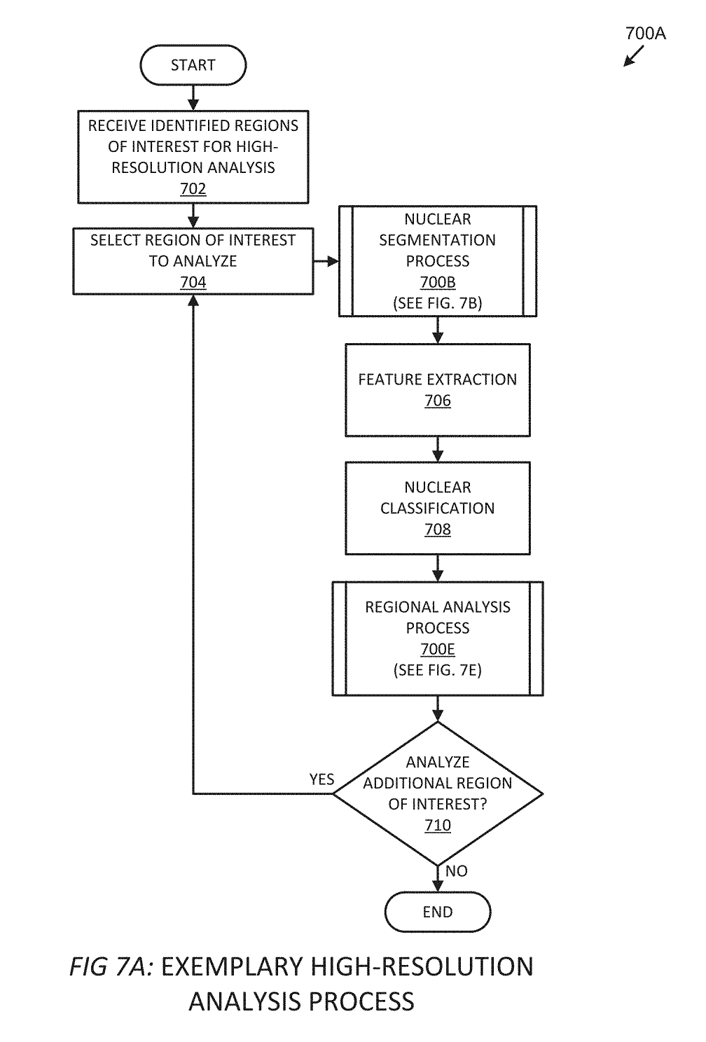

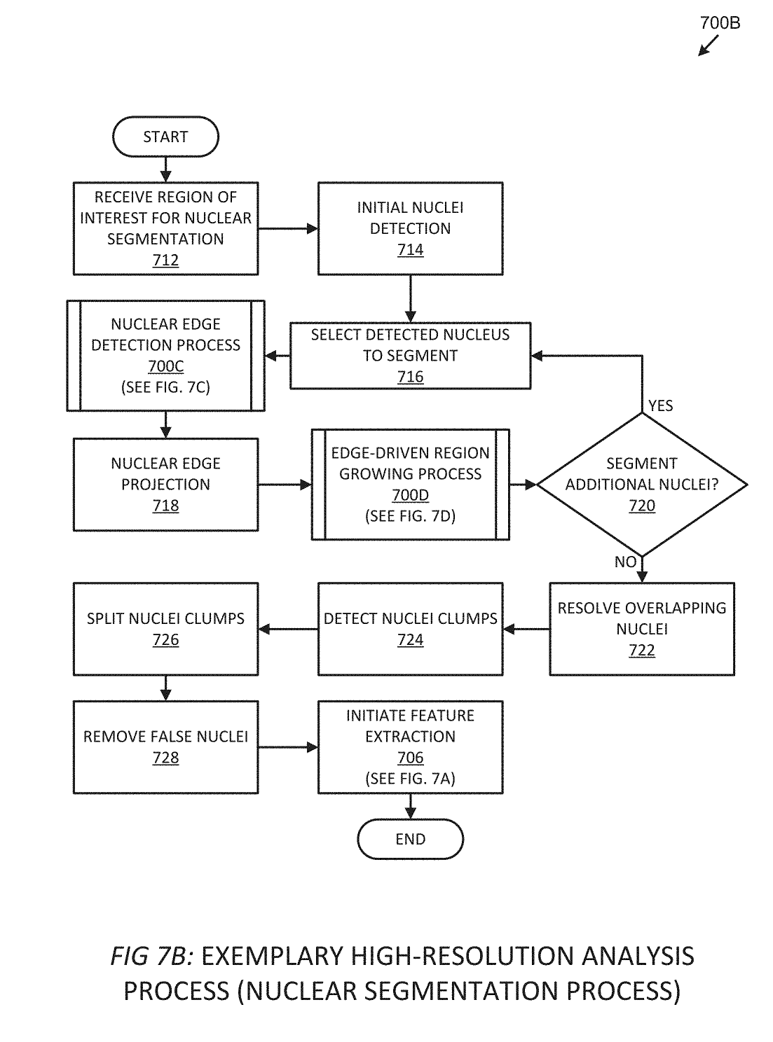

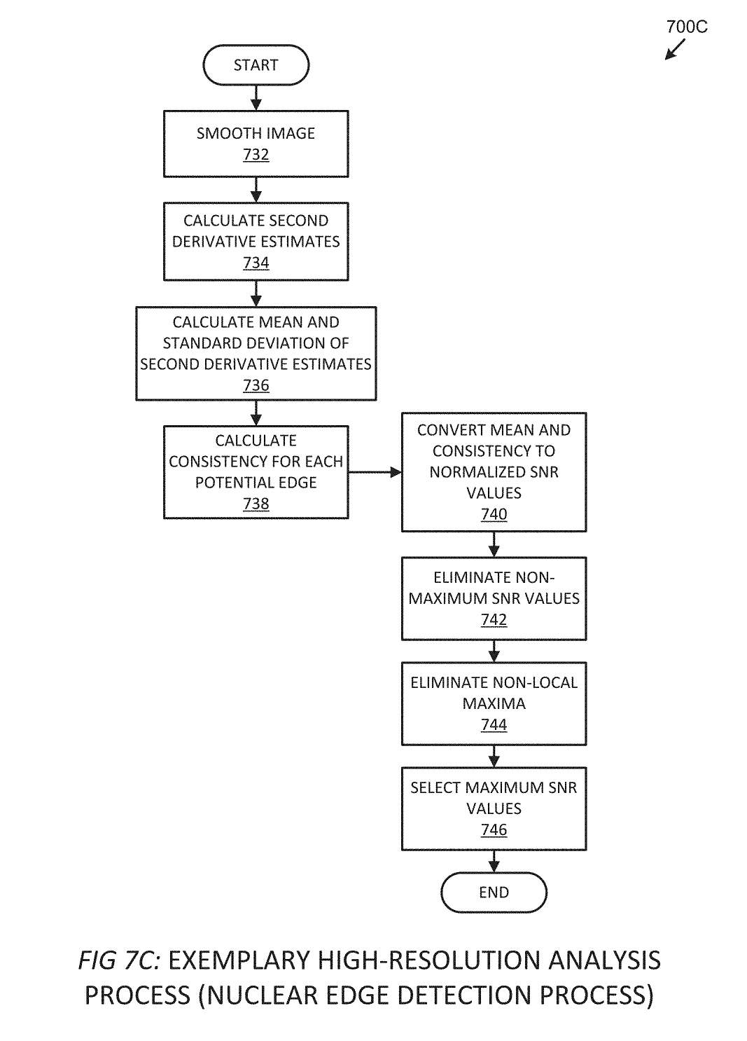

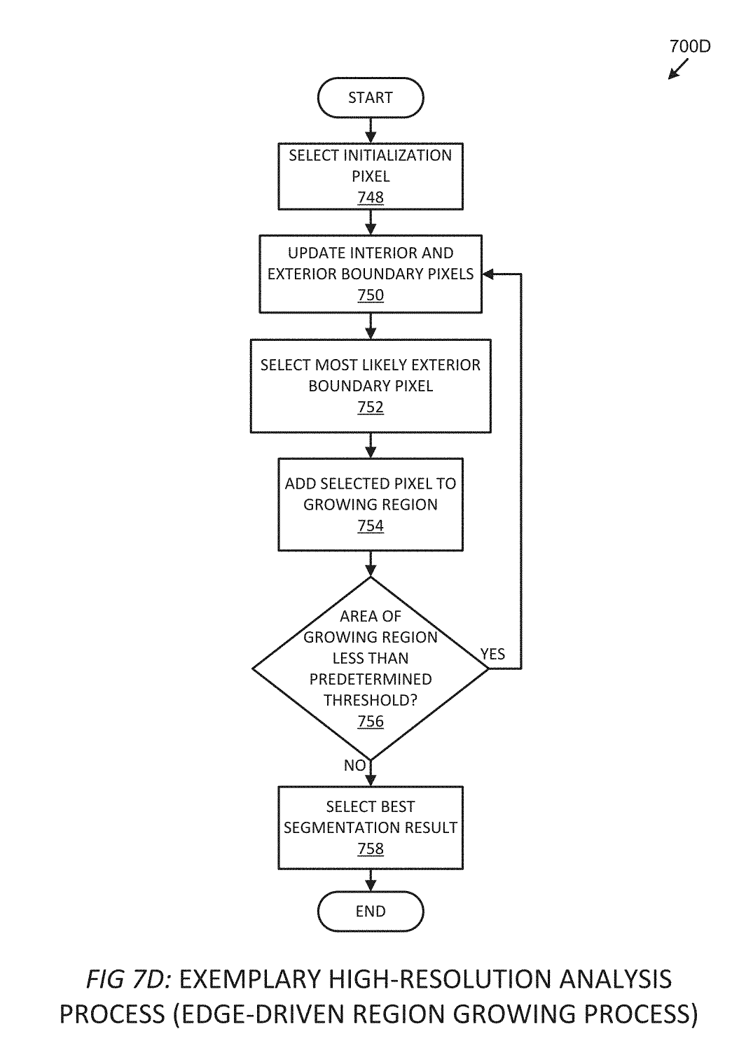

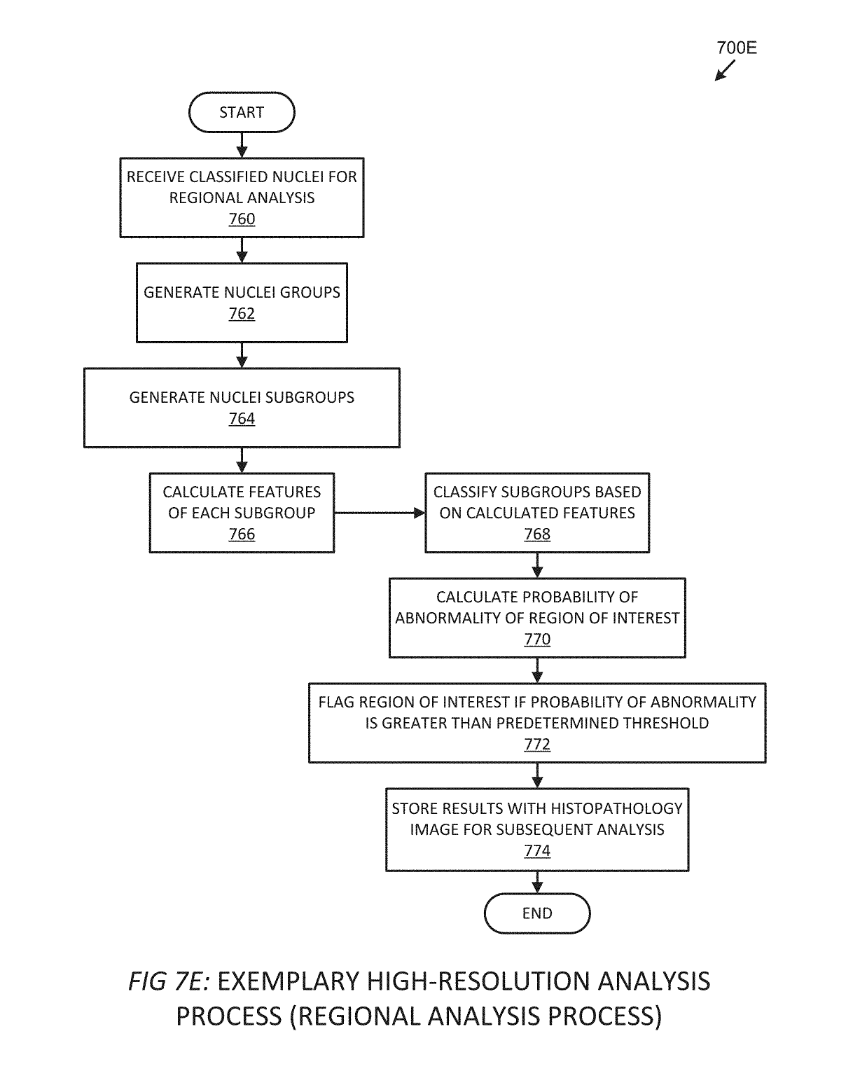

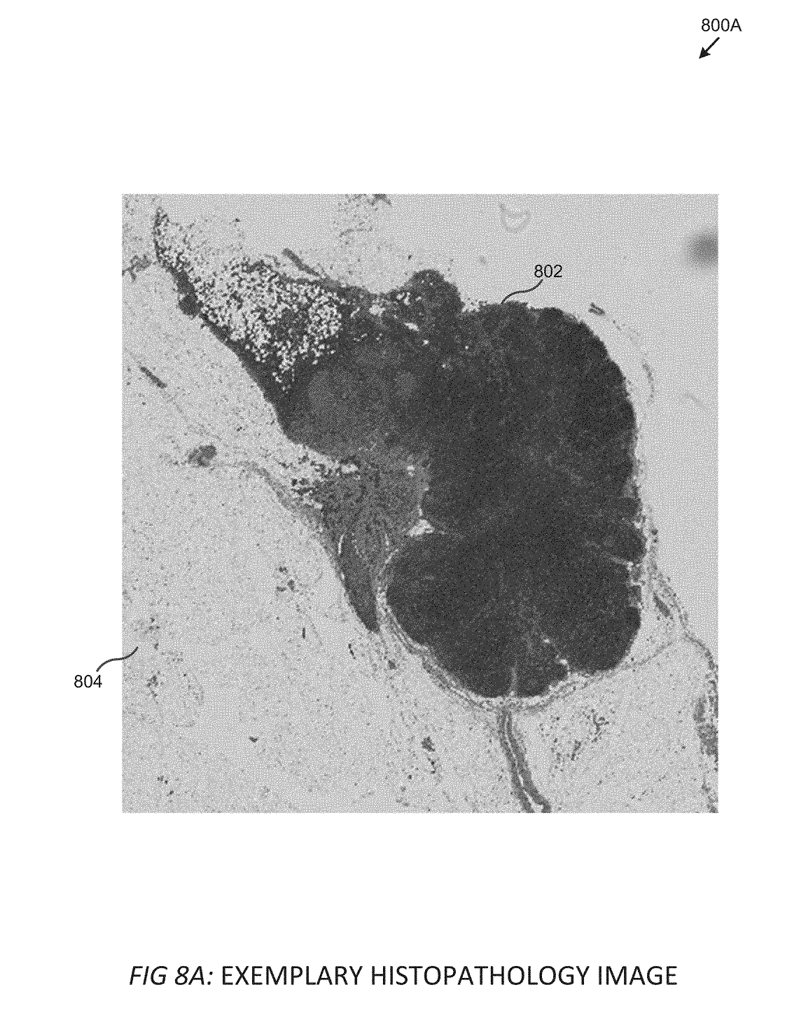

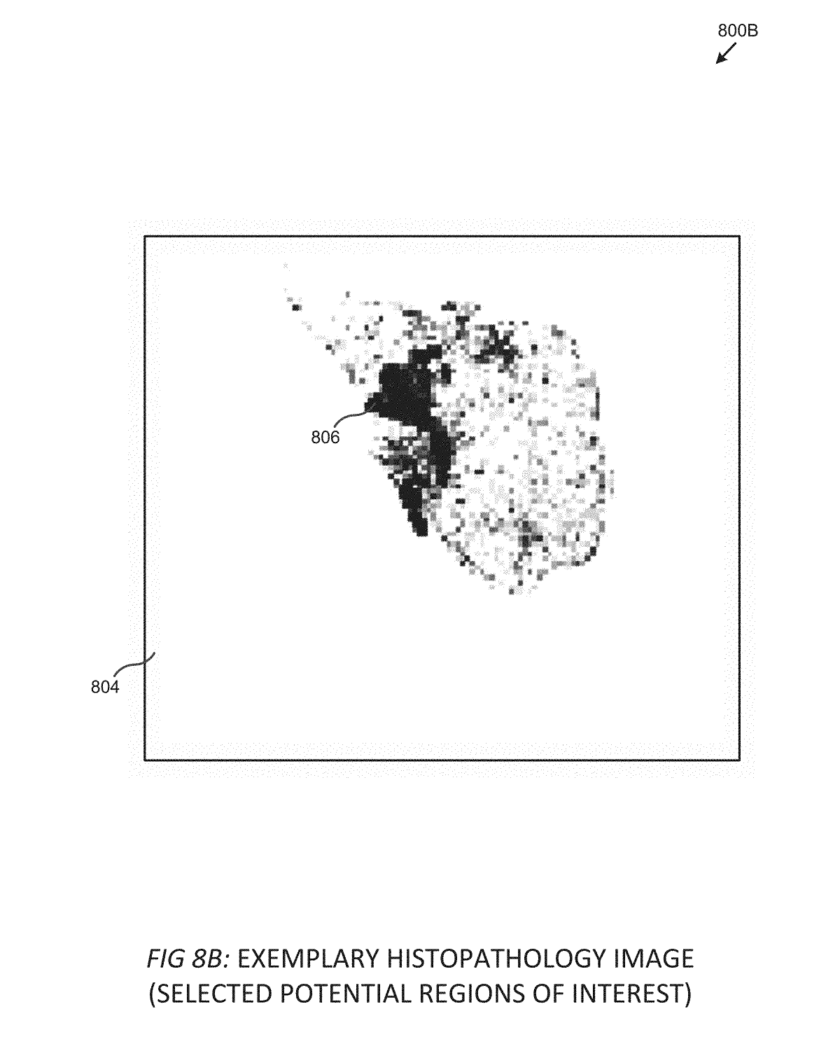

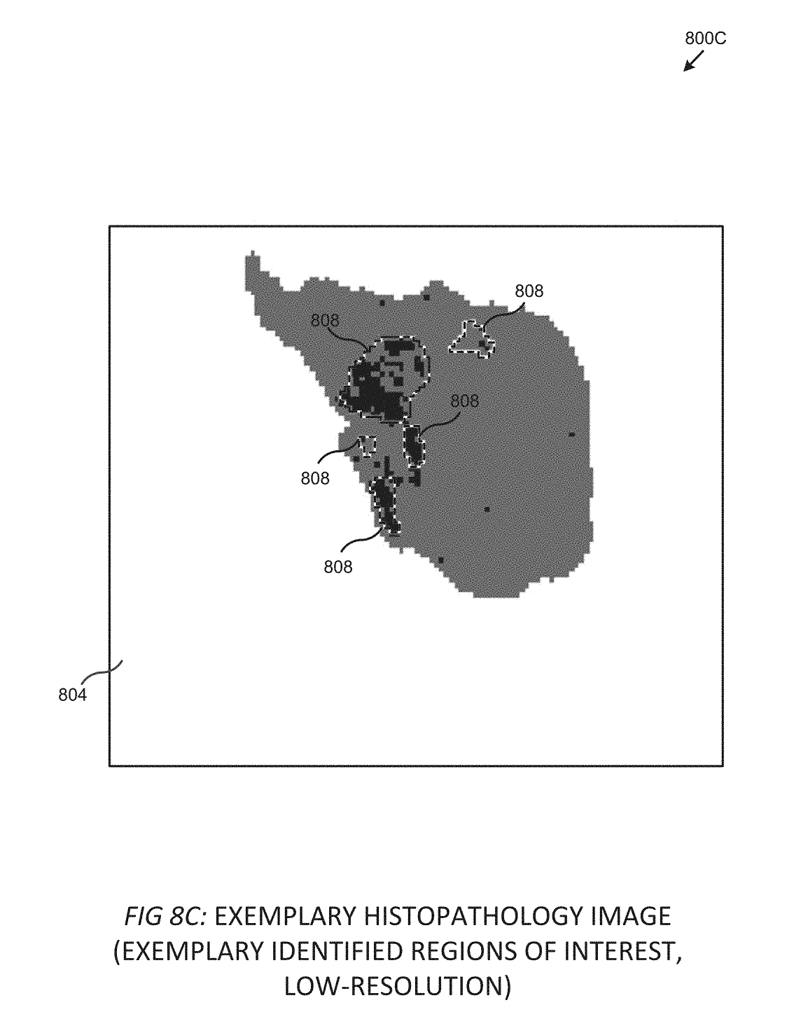

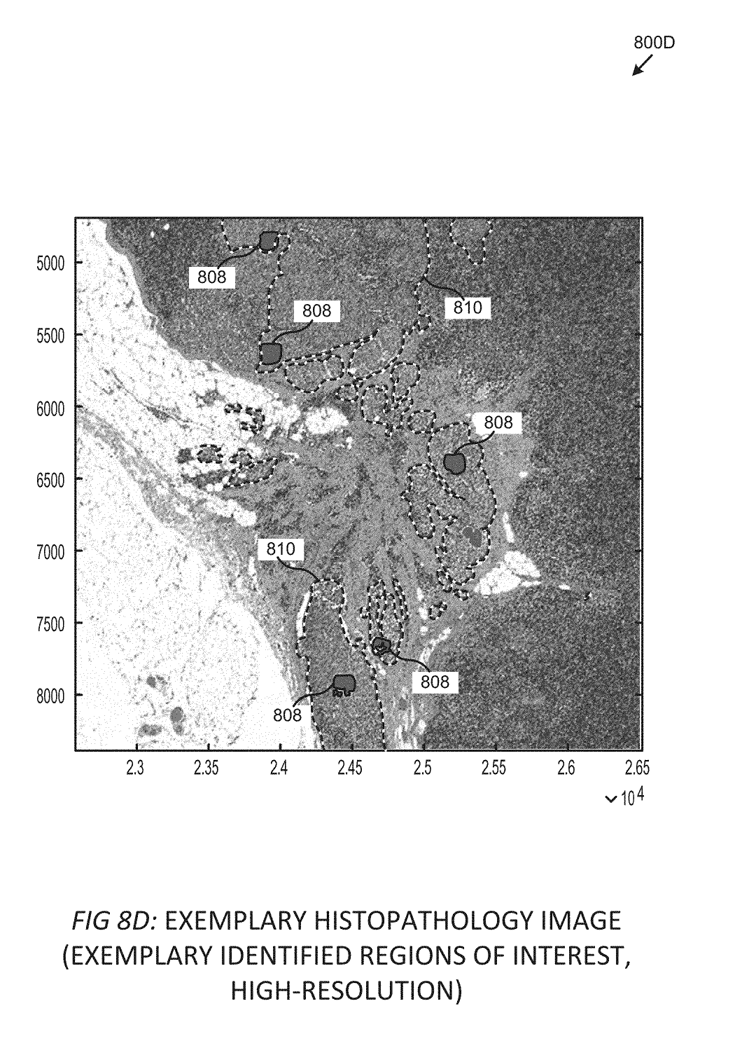

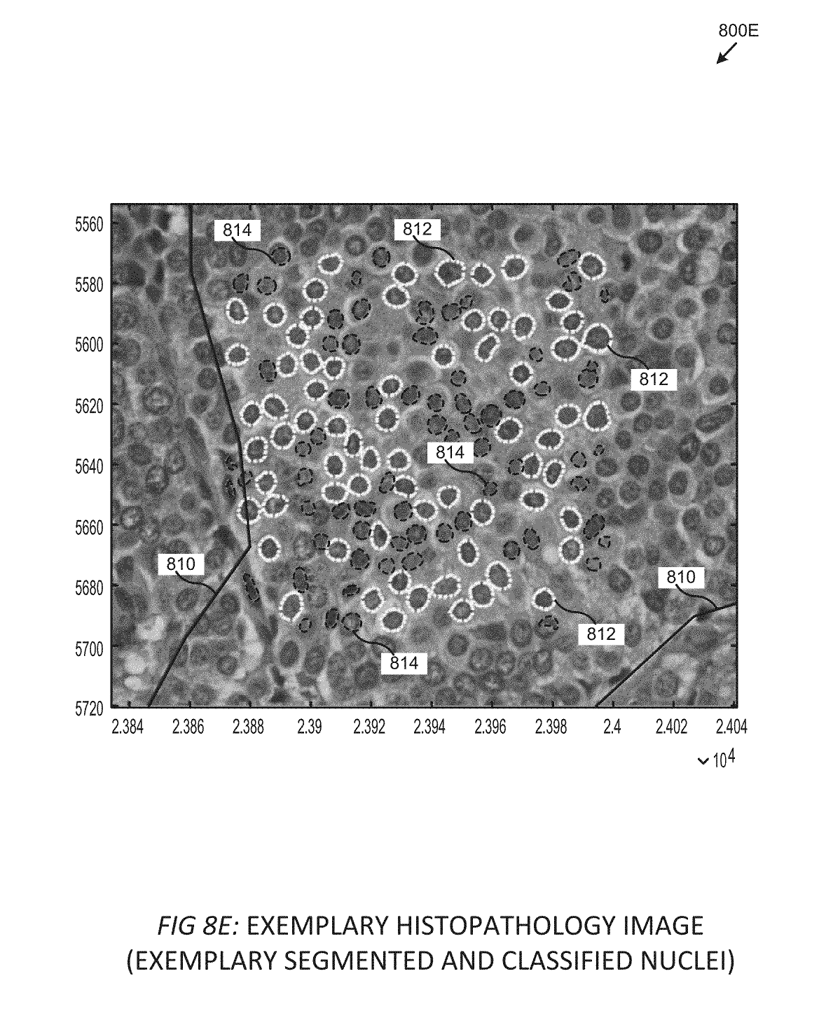

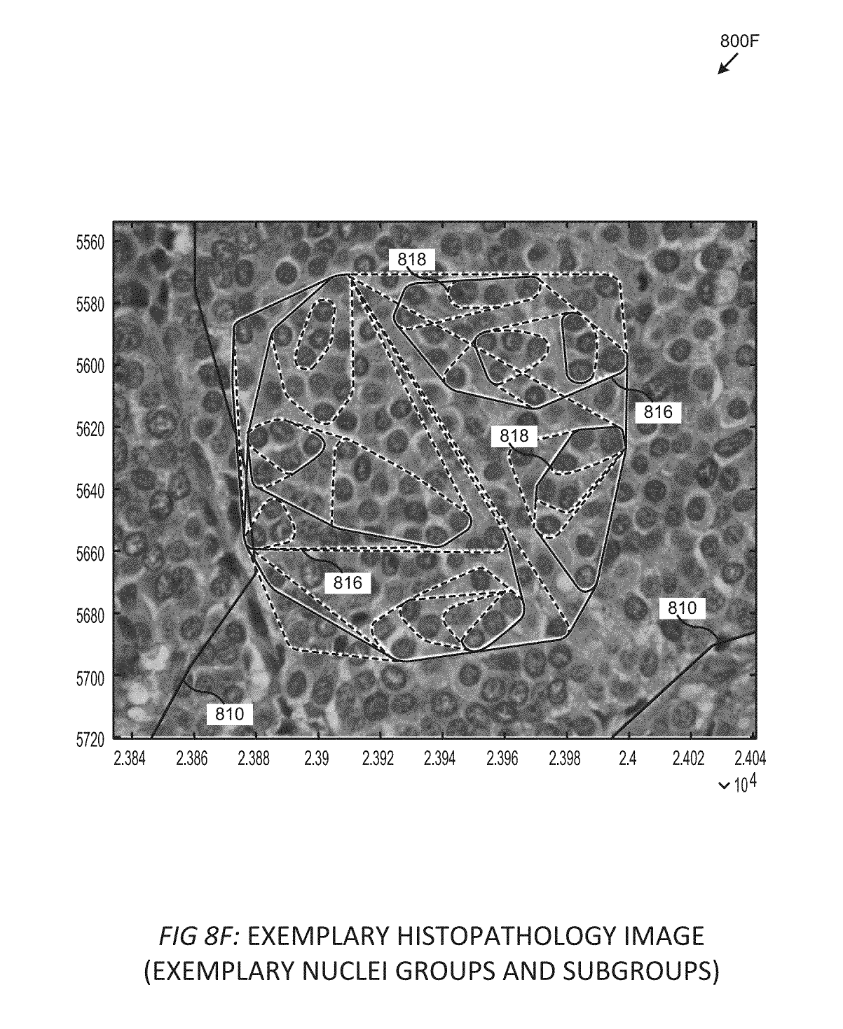

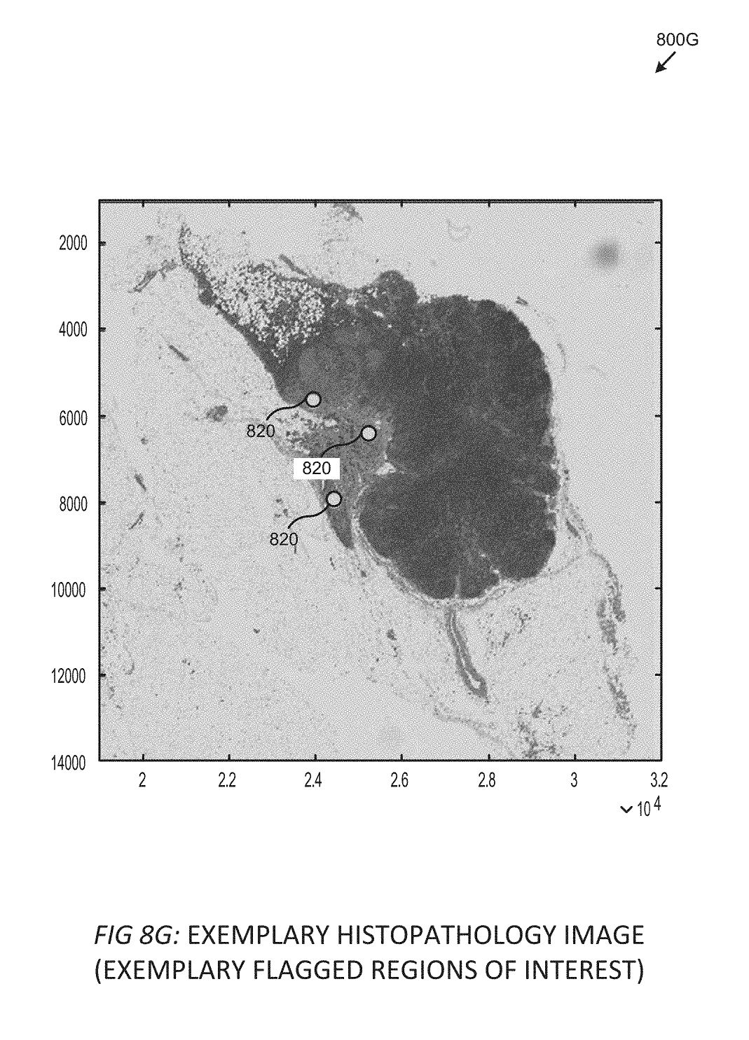

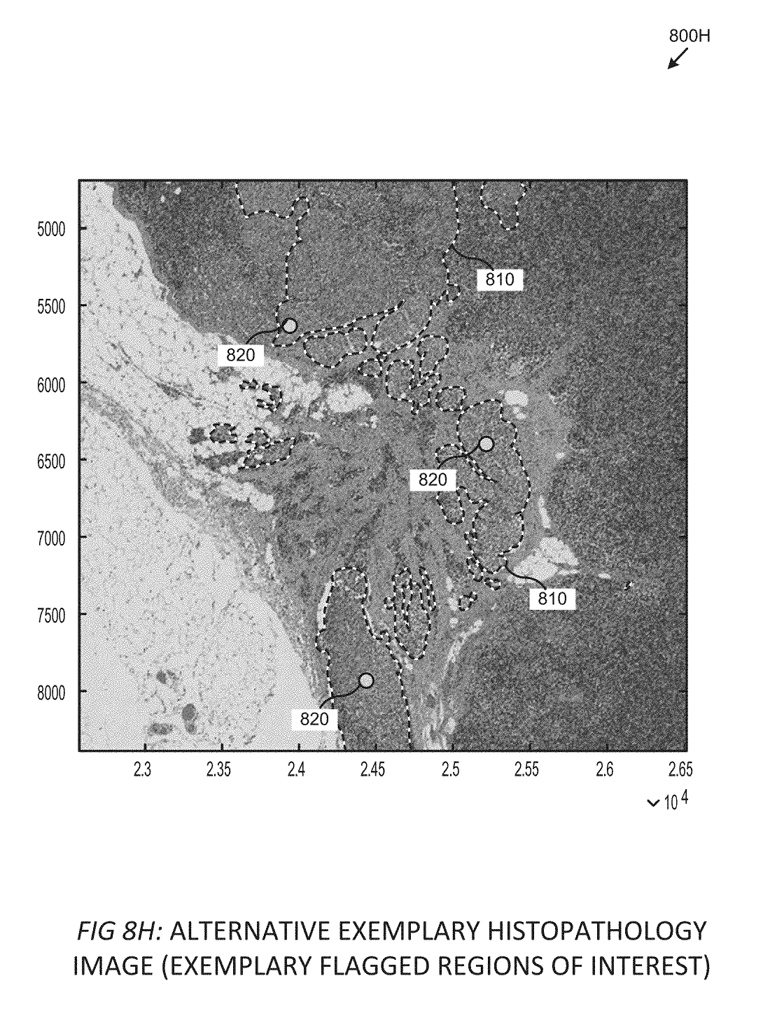

After the histopathology image(s) has been generated, the histopathology image(s) is transmitted to the tissue analysis system 102 for identification of regions of interest. Generally, the tissue analysis system 102 may process multiple histopathology images either concurrently or simultaneously to identify regions of interest in each of the histopathology images. In various embodiments, the identification of regions of interest within a histopathology image comprises the following processes: tissue identification, artifact removal, low-resolution analysis, and high-resolution analysis. In one embodiment, tissue identification is the process by which the tissue analysis system 102 identifies tissue regions (and, in one embodiment, a particular type of tissue) within the histopathology image (e.g., separating the tissue regions from the blank background regions), which will be explained in further detail in association with the description of FIG. 4. Artifact removal, in one embodiment, is the process by which the tissue analysis system 102 removes artifacts (e.g., blurry regions, fingerprints, foreign objects such as dust or hair, etc.) that may have accidentally been included on the tissue slide from the histopathology image (further details of which will be explained in association with the description of FIG. 5). In one embodiment, low-resolution analysis is the process by which the tissue analysis system 102 identifies potential regions of interest, with an emphasis on speed and/or low-resource processing (not necessarily accuracy), for subsequent confirmation as regions of interest based on certain predefined features within the identified tissue (e.g., cellular structures, nuclei patterns, etc.), which will be explained in further detail in association with the description of FIG. 6. High-resolution analysis, in one embodiment, is the process by which the tissue analysis system 102 confirms whether a particular potential region of interest should be considered a region of interest, based on predefined cell nuclei patterns, for subsequent analysis by a professional (further details of which will be explained in association with the description of FIG. 7). In various embodiments, a display of the identified regions of interest (and other parts of the process as disclosed herein) is overlaid as a layer(s) on top of the histopathology image that may be viewed (or removed from view) by the professional (further details of which will be explained in association with the description of FIG. 8).

For example, continuing with the lymph node tissue example, the tissue analysis system 102 processes the histopathology image(s) of the lymph node tissue to identify regions of interest that may contain cancerous cells. Accordingly, the histopathology image(s) undergo a tissue identification process to identify the lymph node tissue within the histopathology image(s) and confirm that the tissue is lymph node tissue and not other tissue (e.g., fat tissue, etc.). Similarly, the histopathology image(s) undergo an artifact removal process to remove any artifacts contained within the histopathology image(s). The histopathology image(s) undergo a low-resolution analysis process to quickly identify potential regions of interest for further analysis during a high-resolution analysis process, during which the tissue analysis system 102 identifies, for subsequent review by a pathologist, regions of interest that may contain cancerous cells.

Thus, in various embodiments, the processed histopathology image with the identified regions of interest is viewed on an electronic computing device 108 by a professional. Generally, the electronic computing device 108 may be any device capable of displaying the processed histopathology image with sufficient resolution so that a professional may confirm whether a region of interest contains a certain predefined abnormality (e.g., computer, laptop, smartphone, tablet computer, etc.). In various embodiments, the professional may view multiple layers of the processed histopathology image as part of the subsequent analysis of the histopathology image. For example, the pathologist may view the lymph node tissue 802 in a view 110 without any of the layers from the process disclosed herein so that the pathologist is not influenced by the identifications made by the tissue analysis system 102 (e.g., in a view 110 as if the pathologist were viewing the original tissue slide 104). Similarly, the pathologist may view the lymph node tissue in a view 112 that shows cell nuclei groups 816 that were identified by the tissue analysis system 102 (as will be discussed in association with the description of FIG. 7). Additionally, the pathologist may view the lymph node tissue in a view 114 that shows identified regions of interest 820 within the histopathology image (as will be discussed in association with the description of FIG. 7). With an understanding of the high-level overview 100 of the tissue analysis system 102, an explanation of an exemplary system architecture of the same may be useful.

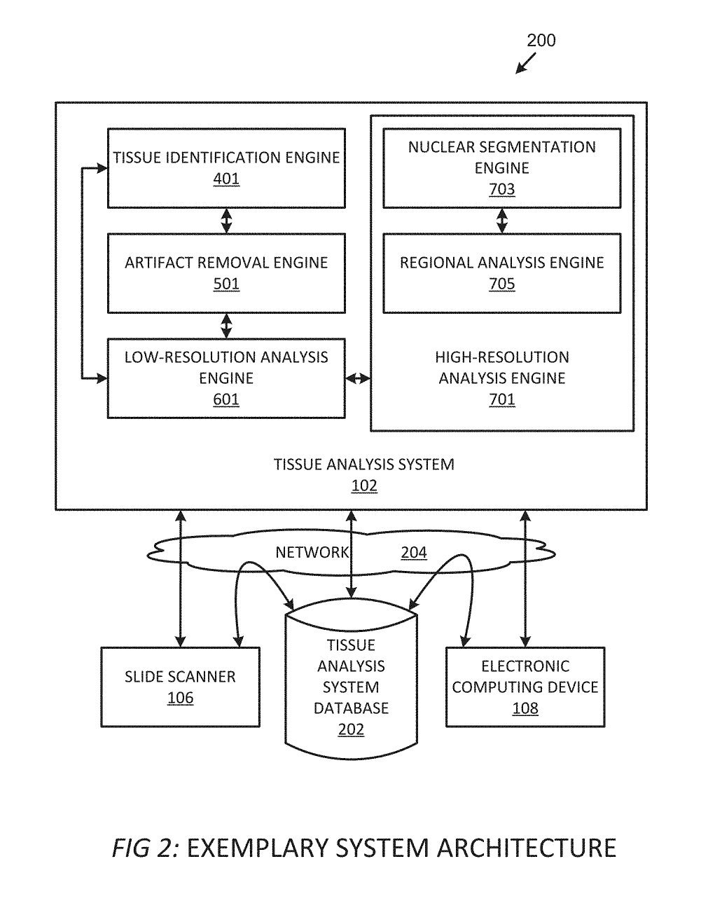

Now referring to FIG. 2, an exemplary architecture 200 of one embodiment of the disclosed system is shown. The exemplary architecture 200 in FIG. 2 is shown for illustrative purposes only and could comprise only one engine, module, or collection of code, etc. In various embodiments, the tissue analysis system 102 is operatively connected to a slide scanner 106, electronic computing device 108, and tissue analysis system database 202 via network 204 to conduct the processes disclosed herein. Generally, network 204 may any connection capable of transferring data between two or more computer systems (e.g., a secure or unsecured connection, Bluetooth, wireless or wired local-area networks (LANs), cell network, the Internet, etc.).

In various embodiments, the slide scanner 106 is any device that is capable of performing the functionality disclosed herein, such as ultra-high resolution scans of many tissue slides 104 at once (e.g., Ultra-Fast Scanner, available from Philips Digital Pathology, Best, Netherlands). In various embodiments, the slide scanner 106 communicates via network 204 with the tissue analysis system 102 and tissue analysis system database 202 to provide histopathology images for processing and storage, respectively.

Generally, the electronic computing device 108 is any device that is capable of performing the functionality disclosed herein and comprises a high-resolution display (e.g., desktop computer, laptop computer, tablet computer, smartphone, etc.). In various embodiments, the electronic computing device 108 communicates via network 204 with the tissue analysis system 102 and tissue analysis system database 202 to view processed histopathology images and, in one embodiment, provide certain administrative functionality with respect to the tissue analysis system 102 (e.g., defining preferences, calibrating, etc.).

Still referring to FIG. 2, the tissue analysis system database 202, in various embodiments, may be any computing device (e.g., desktop computer, laptop, servers, tablets, etc.), combination of computing devices, software, hardware, combination of software and hardware, database (e.g., stored in the cloud or on premise, structured as relational, etc.), or combination of databases that is capable of performing the functionality disclosed herein. In one embodiment, the tissue analysis system database 202 is local to the electronic computing device 108 (e.g., the electronic computing device 108 comprises the tissue analysis system database 202). In other embodiments, the tissue analysis system 102 is virtual or stored in the “cloud.” In various embodiments, tissue analysis system database 202 communicates via network 204 with the tissue analysis system 102, slide scanner 106, and electronic computing device 108 to store processing rules/preferences/algorithms, histopathology images, processed histopathology images, histopathology image libraries, etc.

Generally, the tissue analysis system 102 (and its engines) may be any computing device (e.g., desktop computer, laptop, servers, tablets, etc.), combination of computing devices, software, hardware, or combination of software and hardware that is capable of performing the functionality disclosed herein. In various embodiments, the tissue analysis system 102 may comprise a tissue identification engine 401, artifact removal engine 501, low-resolution engine 601, and high-resolution engine 701. In one embodiment, the tissue identification engine 401 conducts the tissue identification process (further details of which will be discussed in association with the description of FIG. 4) and communicates with the artifact removal engine 501 and low-resolution analysis engine 601. The artifact removal engine 501, in one embodiment, conducts the artifact removal process (further details of which will be discussed in association with the description of FIG. 5) and communicates with the tissue identification engine 401 and low-resolution analysis engine 601. In one embodiment, the low-resolution analysis engine 601 conducts the low-resolution analysis process (further details of which will be discussed in association with the description of FIG. 6) and communicates with the tissue identification engine 401, artifact removal engine 501, and high-resolution analysis engine 701. The high-resolution analysis engine 701, in one embodiment, conducts the high-resolution analysis process (further details of which will be discussed in association with the description of FIG. 7) and communicates with the low-resolution analysis engine 601. In one embodiment, the high-resolution analysis engine 701 comprises the nuclear segmentation engine 703 and the regional analysis engine 705, which communicate with each other to conduct the nuclear segmentation process and regional analysis process, respectively (further details of which will be discussed in association with the description of FIGS. 7B, 7C, 7D, and 7E). To further understand the exemplary architecture 200, an explanation of the tissue analysis process may be helpful.

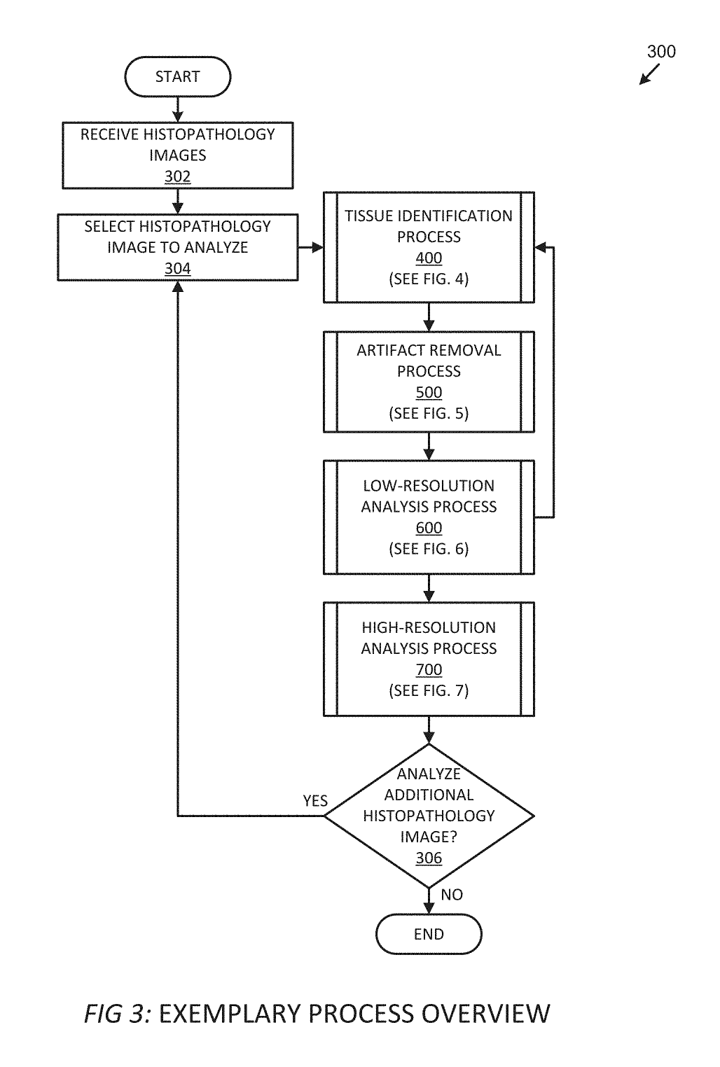

Referring now to FIG. 3, an exemplary overview of a tissue analysis process 300 is shown according to one embodiment of the present disclosure. Generally, the tissue analysis process 300 is the process by which the tissue analysis system 102 (from FIG. 1) processes histopathology images to identify regions of interest that may contain certain predefined abnormalities. As will be understood by one having ordinary skill in the art, the steps and processes shown in FIG. 3 (and those of all other flowcharts and sequence diagrams shown and described herein) may operate concurrently and continuously, are generally asynchronous and independent, and are not necessarily performed in the order shown.

In various embodiments, the tissue analysis process 300 begins at step 302 when the tissue analysis system receives one or more histopathology images. In one embodiment, the histopathology images may come directly from a slide scanner (e.g., slide scanner 106 from FIG. 1), from local/network storage (e.g., tissue analysis system database 202 from FIG. 2), or from some other source (e.g., via email from a third party, etc.). As will occur to one having ordinary skill in the art, the histopathology images may be received as part of a bulk import/batch of histopathology images pertaining to one or more patients. Thus, at step 304, in one embodiment, the system selects a particular histopathology image to analyze/process—this selection may occur automatically according to predefined rules, after selection by a user, etc. Generally, the tissue analysis process 300 may analyze multiple histopathology images concurrently or simultaneously.

In one embodiment, the tissue analysis process 300 continues with the tissue identification process 400, wherein the system identifies tissue regions and, in one embodiment, a particular type of tissue within the histopathology image (further details of which will be explained in association with the description of FIG. 5). Generally, a histopathology image comprises regions of tissue and regions of background corresponding to the absence of tissue. Thus, the tissue identification process 400 separates the tissue regions from the blank background regions so that the system knows which regions to analyze for the presence of predefined abnormalities. In a particular embodiment, the tissue identification process 400 also determines whether the identified tissue corresponds to a particular type of tissue. For example, a histopathology image of lymph node tissue may be processed to identify the tissue from the background and to confirm that the identified tissue is indeed lymph node tissue (and not, for example, adipose tissue).

After the tissue identification process 400, in one embodiment, the system proceeds with the artifact removal process 500 (further details of which will be explained in association with the description of FIG. 5). In various embodiments, the artifact removal process 500 is the process by which the system removes, from the histopathology image, artifacts (e.g., blurry regions, fingerprints, foreign objects such as dust or hair, etc.) that may have accidentally been included on the tissue slide. Generally, the artifact removal process 500 helps limit false-positive identifications of regions of interest. For example, the artifact removal process 500 may remove blurry regions (e.g., regions that are out of focus) from a histopathology image containing lymph node tissue so that those blurry regions are not misidentified as regions of interest (e.g., potentially cancerous cells).

In one embodiment, the tissue analysis process 300 continues with the low-resolution analysis process 600, wherein the system identifies potential regions of interest within the histopathology images (further details of which will be explained in association with the description of FIG. 6). Generally, the low-resolution analysis process 600 identifies potential regions of interest based on certain predefined features within the identified tissue (e.g., cellular structures, nuclei patterns, etc.). In one embodiment, the system calibrates itself by returning to the tissue identification process 400 (e.g., as part of a machine-learning process, the system reviews previously-identified regions of interest that contain the predefined features for which it is analyzing the histopathology images). In various embodiments, the low-resolution analysis process 600 is performed with an emphasis on speed/low-resource processing and not on accuracy. For example, the low-resolution analysis process 600 may quickly identify potential regions of interest (e.g., potentially cancerous cells) within a lymph node tissue histopathology image. In one embodiment, the system only performs the low-resolution analysis process 600 to determine the presence of a predefined abnormality and the remains steps and/or process in FIG. 3 are omitted.

After the low-resolution analysis process 600, in one embodiment, the system proceeds with the high-resolution analysis process 700 (further details of which will be explained in association with the description of FIG. 7). Generally, the high-resolution analysis process 700 is the process by which the system confirms whether a particular potential region of interest should be considered a region of interest, based on predefined cell nuclei patterns of cells shown in the tissue image. In one embodiment, the high-resolution analysis process 700 identifies nuclei within the previously-identified tissue of a particular histopathology image and determines, based on grouping patterns of the identified nuclei, whether the nuclei correspond to a predefined abnormality. For example, the system may determine that a particular group of nuclei within lymph node tissue are potentially cancerous and identify those nuclei as a region of interest. In one embodiment, the high-resolution analysis process 700 is based on predefined patterns of the connective tissue and/or cytoplasm shown in the tissue image. In one embodiment, the system only performs the high-resolution analysis process 700 to determine the presence of a predefined abnormality and the remains steps and/or process in FIG. 3 are omitted.

After the high-resolution analysis process 700, in one embodiment, the system determines, at step 306, whether there are additional histopathology images to process/analyze. If there are additional histopathology images to process/analyze, then the tissue analysis process 300 returns to step 304 and selects a histopathology image to process/analyze. If there are no additional histopathology images to process/analyze, then the tissue analysis process 300 ends thereafter. To further understand the tissue analysis process 300, additional explanation may be useful.

Now referring to FIG. 4, an exemplary tissue identification process 400 is shown according to one embodiment of the present disclosure. Generally, the exemplary tissue identification process 400 is the process by which the tissue analysis system 102 (from FIG. 1) identifies tissue regions from the background of the histopathology image and, in one embodiment, a particular type of tissue within the histopathology image. The identification of tissue and a particular tissue type may, in various embodiments, reduce the area of the histopathology image to be analyzed so that any additional analysis of the histopathology image may be performed more efficiently.

In one embodiment, the exemplary tissue identification process 400 begins at step 402 when the system (e.g., tissue identification engine 401 from FIG. 2) receives the selected histopathology image. Thus, at step 404, in various embodiments, the system determines whether to detect the presence of tissue within the histopathology image or to confirm the particular tissue type of the tissue within the histopathology image based on predefined rules or a selection by a user. If the system determines, at step 404, to detect the presence of tissue within the histopathology image, then the system proceeds to step 406. If, however, the system determines, at step 404, to confirm the particular tissue type of the tissue within the histopathology image, then the system proceeds to step 420.

Thus, at step 406, in one embodiment, the system selects an appropriately-sized image from the histopathology image for the analysis based on predefined criteria. Generally, the histopathology image may be stored as an image pyramid with multiple levels (e.g., the base level is the highest-resolution image and each level above corresponds to a lower-resolution version of the image below it). In one embodiment, the histopathology image may comprise an image with dimensions of 80,000×60,000 pixels, so the system selects a level that is less than 1,000 pixels in each dimension for ease of processing (and to limit the amount of necessary processing). In various embodiments, after selecting the appropriately-sized image, the system converts, at step 408, the selected image into the appropriate color space for the subsequent processing (e.g., a specific organization of colors such as sRGB, CIELCh, CMYK, etc.). As will occur to one having ordinary skill in the art, the appropriate color space may depend on the parameters of the subsequent processing algorithms. For example, a given histopathology image may be stored in sRGB color space, whereas an embodiment of the system may require CIELCh color space, so the system converts the histopathology image to CIELCh color space. In one embodiment, if the appropriately-sized image was created in the appropriate color space, then the system skips step 408 because the conversion is unnecessary.

Still referring to FIG. 4, at step 410, in various embodiments, the system eliminates unscanned areas of the histopathology image from the analysis (e.g., background, non-tissue areas). In one embodiment, the system uses Otsu's method to perform clustering-based image thresholding, thereby removing portions of the histopathology image that are above a certain threshold (e.g., 95/100 on a lightness threshold, etc.), but a person having ordinary skill in the art will recognize that any similar method may be used at step 410. Once eliminated, the pixels are usually excluded from subsequent processing by the system. In various embodiments, at step 412, the system identifies the foreground (e.g., tissue) and background (e.g., non-tissue) of the histopathology image using, for example, a Gaussian mixture model. For example, the system computes the distribution of the chroma values of the remaining pixels within the histopathology image, wherein higher-chroma pixels are considered foreground and lower-chroma pixels are considered background.

At step 414, in various embodiments, when the distribution of foreground or background does not fit a Gaussian mixture model, the system selects a threshold foreground/background value and marks pixels above the threshold as background (e.g., non-tissue) and below the threshold as foreground (e.g., tissue). Generally, steps 410, 412, and 414 perform similar functionality; thus, in one embodiment, the system only performs one of steps 410, 412, and 414 to identify the background (e.g., non-tissue) and foreground (e.g., tissue) regions of the histopathology image. At step 416, in various embodiments, once the tissue and non-tissue regions are identified, a binary layer (alternatively referred to herein as a “mask” or a “tissue mask”) is generated that identifies the tissue and non-tissue regions. Generally, the mask may be refined with a sequence of morphological filters to remove small holes in the mask (which likely should be identified as tissue) or small islands of tissue (which likely should be identified as non-tissue). In one embodiment, the mask is stored with the histopathology image so that subsequent processes may utilize the mask. Thus, at step 418, the system determines whether to confirm the particular tissue type of the tissue within the mask/histopathology image. If the system determines, at step 418, to not confirm the particular type of tissue within the mask, then the system initiates the artifact removal process 500. If, however, the system determines, at step 418, to confirm the particular tissue type of the tissue within the mask, then the system proceeds to step 420.

After determining to confirm the particular tissue type of the tissue within the histopathology image (either at step 404 or 418), then the system proceeds to step 420, wherein the system selects an appropriately-sized image from the histopathology image for the analysis (as performed at step 406). In one embodiment, if an appropriately-size image was previously selected at step 406 (or if the histopathology image is not stored as an image pyramid), then the system selects the previously-selected image or mask (or skips step 420). In various embodiments, after selecting the appropriately-sized image, the system converts, at step 422, the selected image into the appropriate color space for the subsequent processing (e.g., as performed at step 408). In one embodiment, if the appropriately-sized image was already converted into the appropriate color space, then the system skips step 422 because the conversion is unnecessary.

Generally, the system confirms the presence of a particular tissue type within the histopathology image by determining that the tissue comprises a particular expected threshold color value (based on the particular stain used to generate the histopathology image). For example, lymph node tissue, after receiving an H&E stain, is expected to be very blue in color. Accordingly, any tissue that is not very blue is likely not lymph node tissue (e.g., is adipose tissue, tissue of another organ, etc.). In various embodiments, at step 424, the system eliminates non-tissue regions of the histopathology-image from the analysis. Generally, the system may automatically eliminate any pixels within the histopathology image that do not fall within the mask. In one embodiment, the system further selects a particular threshold value and eliminates pixels below that value (e.g., 20^{th} percentile of a particular hue channel). As will occur to one having ordinary skill in the art, the functionality of step 424 may occur in subsequent processes discussed herein even if it is not explicitly described. At step 426, in various embodiments, the system generates an image based on the prototypical color value for the particular tissue by reducing the hue channel for that particular value by that particular value. For example, in an H&E stained histopathology image, the prototypical color value would be a certain blue value; thus, the system would reduce the blue hue channel by that certain blue value to generate an image with more contrast. In various embodiments, at step 428, the system applies a filter (e.g., a 2D order statistic filter) to the generated image to determine the number of pixels within a certain area that are of a certain color value (e.g., at least 10% of the pixels within a 0.32 mm diameter circle are of the expected value). Thus, at step 430, the system selects a threshold value to which to compare all of pixels that pass through the filter.

Accordingly, at step 432, a binary mask is generated with all of the pixels within the threshold and the mask is refined using morphological filters to remove isolated pixels, wherein the mask identifies the particular tissue type. Generally, this mask is stored with the histopathology image so that subsequent processes may utilize the mask. After storing the mask, in various embodiments, the system initiates the artifact removal process 500. In one embodiment, after initiating the artifact removal process 500, the exemplary tissue identification process 400 ends thereafter.

Referring now to FIG. 5, an exemplary artifact removal process 500 is shown according to one embodiment of the present disclosure. Generally, the exemplary artifact removal process 500 is the process by which the tissue analysis system 102 (from FIG. 1) removes artifacts (e.g., fingerprints, dust, blurry regions, etc.) from the histopathology image. The removal of artifacts may, in various embodiments, reduce errors in the subsequent processes that could result from the presence of the artifacts.

In one embodiment, the exemplary artifact removal process 500 begins at step 502 when the system (e.g., artifact removal engine 501 from FIG. 2) receives the selected histopathology image. Thus, at step 504, in various embodiments, the system detects artifacts within the histopathology image for removal (e.g., based on dissimilarities in color/shape of the artifacts in comparison to the surrounding tissue). These artifacts may include physical objects that were improperly include on the tissue slide (e.g., fingerprints, hair, dust, etc.) or blurry/out-of-focus regions that occurred from poor slide preparation (e.g., trapped liquids, smudges on the slide itself, etc.). Thus, at step 506, the system determines whether to remove blurry regions from the histopathology image.

If, at step 506, the system determines that it should remove blurry regions (because blurry regions were identified at step 504 and/or according to a predefined rule), then the system proceeds at step 508 to extract a predetermined color channel from the histopathology image (e.g., red, etc.) to improve the contrast of the histopathology image. At step 510, the system divides the histopathology image into regions of predetermined size (e.g., 100×100 pixels). Thus, at step 512, the system calculates the sharpness of each region by calculating a direction of the edge within the region, determining the pixels that correspond to that edge, calculating a thickness of each edge pixel (e.g., using a Taylor approximation, etc.), and calculating the sharpness of the region (e.g., the inverse of the median of the edge pixels thickness, etc.). Based on the sharpness, the system, at step 514, classifies regions below a predetermined threshold as blurry and removes them from the mask/subsequent analysis. After classifying regions as blurry, the system, in one embodiment, initiates the low-resolution analysis process 600 (or, not shown in FIG. 5, returns to step 516 to remove other identified artifacts).

If, however, at step 506, the system determines that it should not remove blurry regions (because none exist within the histopathology image and/or according to a predefined rule indicating, for example, certain areas from which to remove blurry regions, etc.), then the system proceeds at step 516 to remove the other identified artifacts from the histopathology image using image processing techniques similar to those used in steps 508-514. After removing the other identified artifacts, the system, in various embodiments, initiates the low-resolution analysis process 600. In one embodiment, after initiating the low-resolution analysis process 600, the exemplary artifact removal process 500 ends thereafter.

Now referring to FIG. 6, an exemplary low-resolution analysis process 600 is shown according to one embodiment of the present disclosure. Generally, the exemplary low-resolution analysis process 600 is the process by which the tissue analysis system 102 (from FIG. 1) quickly identifies potential regions of interest based on certain predefined features within the identified tissue (e.g., cellular structures, nuclei patterns, etc.). The identification of potential regions of interest may, in various embodiments, reduce the processing time for the remaining processes and minimize the expenditure of processing resources during the same.

In one embodiment, the exemplary low-resolution analysis process 600 begins at step 602 when the system (e.g., low-resolution analysis engine 601 from FIG. 2) receives the selected histopathology image. Thus, at step 604, in various embodiments, the system determines whether to calibrate the low-resolution analysis engine so that its algorithms recognize the types of abnormalities for which the tissue analysis process is to identify. In one embodiment, the calibration (e.g., steps 606-612) may occur prior to the processing of histopathology images by the tissue analysis system and may occur only once for each type of abnormality. In various embodiments, the system determines to calibration the low-resolution analysis engine based on predefined rules (e.g., calibrate once during a predetermined time period, etc.) or a decision by a user. If the system determines, at step 604, to calibrate the low-resolution analysis engine, then the system proceeds, in one embodiment, to step 606, wherein the system requests (from the tissue analysis system database 202, third party systems, etc.) histopathology images containing abnormalities, previously marked/identified by a professional, representative of the type of abnormality for which the system is searching (e.g., examples of the types of abnormalities for which the system is searching). Thus, at step 608, in various embodiments, the system receives the representative histopathology images and processes those images through the tissue identification process 400 and artifact removal process 500 to generate images that the low-resolution analysis engine can easily process itself. In various embodiments, at step 610, the system calculates the texture features (e.g., energy, entropy, homogeneity, correlation, etc.) of random regions within the representative histopathology images (both within and outside of regions that have been previously identified as corresponding to the relevant type of abnormality). In one embodiment, texture features may comprise a set of predetermined metrics to be calculated in a particular histopathology image to quantify the perceived texture of the image (e.g., local binary patterns, gray level run length, FFT features, Gabor filters, histogram analysis features, wavelet analysis, etc.). Thus, at step 612, in various embodiments, the system uses the calculated texture features to generate baseline abnormality thresholds against which histopathology images are compared and calibrates the low-resolution analysis engine based on those thresholds. The system, in one embodiment, returns to step 604.

If the system determines, at step 604, not to calibrate the low-resolution analysis engine, then the system proceeds, in various embodiments, to step 614, wherein the system uniformly splits the histopathology image (e.g., the particular tissue type mask) into potential regions of interest (e.g., 100×100 pixel squares) that will each be analyzed to determine whether they potentially contain abnormalities. In one embodiment, the system splits the histopathology image in random, non-uniform potential regions of interest. Thus, at step 616, in various embodiments, the system calculates the texture features within each of the potential regions of interest. In various embodiments, at step 618, the system classifies the texture features by calculating a confidence metric for each potential region of interest that indicates the likelihood that a particular region of interest comprises the abnormality (e.g., how similar the calculated text features are to the texture features of the representative histopathology images from calibration). Accordingly, at step 618, in various embodiments, the system identifies regions of interest for high-resolution analysis by generating a “map” of the confidence metrics for the histopathology image, eliminating any false positives/other outliers of the confidence metrics for regions of interest as compared to the surrounding regions of interest, identifying regions of interest that comprise local maximums of the confidence metrics within the histopathology image, using those local maximums as seed points to grow/generate/identify particular regions of interest that should be analyzed using the high-resolution analysis process (e.g., assessing the size, shape, and confidence of each identified region of interest). In one embodiment, the system attempts to identify the smallest possible number of regions of interest corresponding to the smallest number of the largest abnormalities that were potentially identified (e.g., three regions of interest corresponding to three large tumors instead of six regions of interest corresponding to six small tumors, wherein the three large tumors comprise the six small tumors). Thus, the system initiates the high-resolution analysis process 700 to confirm that the identified regions of interest comprise the particular abnormality.

Referring now to FIG. 7 (consisting of FIGS. 7A, 7B, 7C, 7D, and 7E), an exemplary high-resolution analysis process 700 is shown according to one embodiment of the present disclosure. Generally, the exemplary high-resolution analysis process 700 is the process by which the tissue analysis system 102 (from FIG. 1) confirms whether a particular identified region of interest comprises a certain abnormality (e.g., cancerous cells, tumor tissue, etc.) based on analysis of the nuclei within that region of interest. FIG. 7A illustrates an overview 700A of the exemplary high-resolution analysis process 700 according to one embodiment of the present disclosure. FIG. 7B illustrates an exemplary nuclear segmentation process 700B according to one embodiment of the present disclosure, wherein nuclei are detected within the region of interest. FIG. 7C illustrates an exemplary nuclear edge detection process 700C according to one embodiment of the present disclosure, wherein the edge of a nucleus is determined/detected (as part of the exemplary nuclear segmentation process 700B). FIG. 7D illustrates an exemplary edge-driven region growing process 700D according to one embodiment of the present disclosure, wherein the detected edge of a nucleus is used to identify the shape of the entire nucleus (as part of the exemplary nuclear segmentation process 700B). FIG. 7E illustrates an exemplary regional analysis process 700E according to one embodiment of the present disclosure, wherein the presence of a certain abnormality within a region of interest is confirmed based on analysis of the identified nuclei within the same.

Referring now to FIG. 7A, in various embodiments, the exemplary high-resolution analysis process 700A begins at step 702 when the system (e.g., high-resolution analysis engine 701 from FIG. 2) receives the one or more identified regions of interest from a given histopathology image. Thus, in one embodiment, at step 704, the system selects a particular region of interest to analyze. In various embodiments, the system processes the selected region of interest through the nuclear segmentation process 700B (further details of which will be explained in association with the description of FIG. 7B), wherein the system automatically identifies the nuclei within the region of interest. After the nuclear segmentation process 700B, the system proceeds, at step 706, to extract texture features from the region of interest (e.g., characteristics of the nuclei within a region of interest). For example, the system may extract features using local binary patterns, gray level run length, FFT features, Gabor filters, SGLDM features, histogram analysis features, and/or wavelet analysis on one or more color channels to segment the data within the region of interest into a representative (and more manageable) data set for subsequent processing (and to assist in classifying the nuclei). Similarly, at step 708, in various embodiments, the system classifies the nuclei within the region of interest for subsequent processing, generally determining whether a particular nucleus is abnormal or benign according to certain predefined rules/metrics (e.g., particular shape, size, texture feature, etc. of the nuclei). Accordingly, the system processes the classified nuclei through the regional analysis process 700E (further details of which will be explained in association with the description of FIG. 7E), wherein the system specifically identifies groups/clusters of nuclei that may comprise the abnormality according to certain predefined rules/metrics. Generally, after the regional analysis process 700E, the high-resolution analysis of a particular region of interest is complete; thus, at step 710, in one embodiment, the system determines whether to analyze an additional region of interest. If the system determines, at step 710, to analyze an additional region of interest, then the system returns to step 704 and selects an additional region of interest for analysis. If, however, the system determines, at step 710, not to analyze an additional region of interest, then the exemplary high-resolution analysis process 700 ends thereafter. In one embodiment, the system analyzes identified regions of interest until a predefined number of nuclei groups corresponding to the abnormality have been identified, and then, the system may send the histopathology image to a professional or flag the histopathology image as comprising the abnormality. In one embodiment, the system analyzes identified regions of interest until all identified regions of interest have been analyzed. To further understand the exemplary high-resolution analysis process 700, a description of the nuclear segmentation process 700B may be helpful.

Now referring to FIG. 7B, in various embodiments, an exemplary nuclear segmentation process 700B is shown, wherein individual nuclei are identified and defined/segmented within a region of interest. The exemplary nuclear segmentation process 700B begins at step 712 when the system (e.g., nuclear segmentation engine 703 from FIG. 2) receives the selected region of interest. Thus, in one embodiment, at step 714, the system initially/preliminarily detects nuclei within the region of interest. In various embodiments, the system uses a combination of blob detection (e.g., a multiscale Laplacian of Gaussian approach, wherein the center of the Laplacian of Gaussian operators are indicated as nuclei) and a curvature-based approach (e.g., an analysis of the curvature of the intensity image to locate centers of curvatures, which indicate nuclei) to initially detect nuclei (or a seed pixel within each nucleus, which will be used to define the shape of the nucleus in subsequent processes). Generally, other approaches may be used to detect nuclei within the region of interest; regardless, in one embodiment, the system reduces the number of detected nuclei (e.g., merging nuclei together that are not separated by an edge, etc.) so that redundant nuclei are eliminated from analysis.

At step 716, in various embodiments, the system selects a particular nucleus to segment/further define its shape. Accordingly, the system processes the selected nucleus through the nuclear edge detection process 700C (further details of which will be explained in association with the description of FIG. 7C), wherein the system detects the edge (e.g., nuclear envelope) of the nucleus in one or more locations so that the system may define the edge of the nucleus. At step 718, in various embodiments, the system projects the edge of the nucleus from the detected one or more locations based on a circular model that decreases in confidence from the location to the opposite side of the circle so that a map is generated comprising these weighted projections. In one embodiment, the edge is projected in multiple types (e.g., first derivative edge, positive second derivative edge, and negative second derivative edge) to increase the confidence in the projection. Generally, the system processes the projected edges through the edge-drive region growing process 700D (further details of which will be explained in association with the description of FIG. 7D), wherein the system accurately defines/segments the shape of the nucleus. Thus, at step 720, the system determines whether to segment another nucleus. If the system determines, at step 720, to segment additional nuclei (e.g., because additional nuclei are present within the region of interest), then the system returns to step 716 to do so. If, however, the system determines, at step 720, to not segment additional nuclei, then the system proceeds at step 722.

In various embodiments, at step 722, the system resolves/eliminates overlapping nuclei that may occur because the system accidentally segmented multiple nuclei from the same singular nucleus in the histopathology image. As will occur to one having ordinary skill in the art, because nuclei are segmented at seed pixels, one or more of the segment nuclei may overlap. As nuclei in actuality do not overlap, the conflict of the overlapping nuclei may be resolved to increase the accuracy of the system. Thus, in one embodiment, a fitness score for each overlapping nucleus (e.g., a combination of size score indicating whether the size of the detected nucleus is appropriate, shape score indicating whether the shape of the detected nucleus is appropriate, and edge strength at the detected edge indicating how likely the edge has been detected) is calculated to indicate which nucleus should be retained. In one embodiment, to confirm the correction resolution, the system masks out the pixels of the retained nucleus and conducts steps 716 through 720 again on the eliminated seed pixel to determine whether an entire nucleus may be segmented from that point without the masked pixels (e.g., if it cannot be done, then the resolution was correct). In various embodiments, at step 724, the system detects nuclei clumps (e.g., detected nuclei that are likely larger and more heterogeneous than other detected nuclei because they contain multiple nuclei) that may reduce the accuracy of subsequent processes. Generally, regions with the least probability of being a singular nucleus are identified as potential nuclei clumps. Thus, at step 726, in one embodiment, the system attempts to split images of nuclei clumps into their individual nuclei using a hypothesis-driven model. In one embodiment, all potential nuclei clump splits (e.g., hypotheses) are evaluated using a multiseed approach to the edge-driven region growing process 700D, wherein multiple seed pixels are grown at the same time (e.g., thereby competing for pixels, instead of overlapping to form the clump). The most probable nuclei clump split, in one embodiment, is selected for further processing. Accordingly, at step 728, the system removes false nuclei from the analysis. In various embodiments, nuclei may be unintentionally detected in other cell structures (e.g., stroma, etc.). Generally, these nuclei have irregular shape and color, so the system identifies (e.g., compared to all of the other segmented nuclei) the nuclei that are not the expected shape/color. After removing false nuclei, the system initiates feature extraction (at step 706 from FIG. 7A), and the exemplary nuclear segmentation process 700B ends thereafter. To further understand the exemplary nuclear segmentation process 700B, a description of the exemplary nuclear edge detection process 700C and exemplary edge-driven region growing process 700D may be useful.

Referring now to FIG. 7C, in various embodiments, an exemplary nuclear edge detection process 700C is shown, wherein the edge of a nucleus is detected to identify/segment the nucleus. The exemplary nuclear edge detection process 700C begins at step 732 when the system smooths the image to reduce noise that is likely to increase errors in the processing. As will occur to one having ordinary skill in the art, the system may use any standard technique to smooth the image (e.g., convolution with a low-pass filter, median filtering, anisotropic diffusion, bilateral filtering, etc.). In various embodiments, at step 734, the system calculates the second derivative estimates of the smoothed image to begin detecting the nuclear edge (e.g., using a sampling matrix). In one embodiment, the system uses an arc-shaped sampling matrix at each point within the image to generate second derivative estimates along a potential arc/edge (e.g., based on the brightness of the pixels surrounding a particular pixel). Thus, at step 736, in various embodiments, the system calculates the mean and standard deviation of the second derivative estimates for each possible arc length of a potential arc from the sampling matrix. Similarly, at step 738, in one embodiment, the system calculates the consistency (e.g., mean divided by the standard deviation) for each possible arc length of a potential arc. At step 740, in various embodiments, the system converts the calculated means and consistencies into normalized signal to noise ratio (e.g., “SNR”) values using a cumulative distribution function. Generally, at step 742, for each potential point and rotation angle along an arc, the system retains the maximum SNR value. Similarly, at step 744, the system then eliminates non-local maxima SNR values (e.g., SNR values that are less than any SNR value that is either orthogonally adjacent in image space or adjacent in angle space). Further, at step 746, the system selects the maximum SNR values across an angle so that an edge has been defined with the angle, length, and radius of curvature for each point within the edge known. Thus, the exemplary nuclear edge detection process 700C ends thereafter.

Now referring to FIG. 7D, in various embodiments, an exemplary edge-driven region growing process 700D is shown, wherein the shape of a nucleus is determined to identify/segment the nucleus. The exemplary edge-driven region growing process 700D begins at step 748 when the system selects an initialization pixel from which to grow the shape of the nucleus (e.g., the system determines the shape of a particular nucleus by selecting an initial starting pixel that is believed to be within the nucleus, based on the detected edges from the nuclear edge detection process 700C and step 718 and the initial nuclei detection from step 714, and adds pixels to that initial pixel until the shape of that nucleus has been determined). Generally, the system monitors four sets of pixels: interior region pixels (e.g., those pixels within the growing region that have no neighboring pixels that belong to the exterior/exterior boundary pixels), interior boundary pixels (e.g., those pixels within the growing region that have neighboring pixels that belong to the exterior boundary pixels), exterior boundary pixels (e.g., those pixels outside of the growing region, in the background, that have neighboring pixels that belong to the interior boundary pixels), and exterior pixels (e.g., those pixels outside of the growing region, in the background, that have no neighboring pixels that belong to the interior/interior boundary pixels). Accordingly, in one embodiment, the system selects pixels within the set of exterior boundary pixels and adds them to the set of interior boundary pixels until the growing region exceeds a predetermined threshold (e.g., expected size of the nucleus, number of pixels within the set of interior region pixels, etc.). At step 750, in one embodiment, the system determines which pixels belong to each of the four sets of pixels and statistically calculates the fitness of the growing region and background (e.g., the accuracy of the current growing region and background). In various embodiments, at step 752, the system selects, using a probabilistic approach, the most likely exterior boundary pixel to be added to the set of interior boundary pixels (e.g., based on multivariate normal distribution intensities of the growing region and the background). Thus, the system, at step 754, in various embodiments, adds the selected pixel to the set of interior boundary pixels. At step 756, the system determines whether the growing region is less than a predetermined threshold. Generally, as will occur to one having ordinary skill in the art, the predetermined threshold may correspond to a minimum, maximum, or average expected size of a nucleus for a particular tissue type. If the growing region is less than a predetermined threshold, then, in one embodiment, the system returns to step 750. If, however, the growing region is larger than the predetermined threshold, then, in one embodiment, the system proceeds at step 758, wherein the best segmentation result is selected. In one embodiment, the system selects the segmentation result with the best fitness value (from step 750). Thus, the exemplary edge-driven region growing process 700D ends thereafter. To further understand the exemplary high-resolution analysis process 700, a description of the exemplary regional analysis process 700E may be helpful.

Referring now to FIG. 7E, in various embodiments, an exemplary regional analysis process 700E is shown, wherein the system specifically identifies groups/clusters of nuclei that may comprise the abnormality according to certain predefined rules/metrics. The exemplary regional analysis process 700E begins at step 760 when the system (e.g., regional analysis engine 705 from FIG. 2) receives the classified nuclei. Thus, in one embodiment, at step 762, the system generates nuclei groups based on the classified nuclei (e.g., a group of one or more nuclei, grouped together based on physical proximity, shape, etc.). These groups of classified nuclei permit the system to more reliably identify abnormalities. For example, in one embodiment, the system creates a minimum distance spanning tree based on one or more nuclear features and abnormal probabilities (e.g., using the nuclei as nodes and the distance between the nuclei's centroids as the distance metric) that contains groups of nuclei that have sufficiently high abnormality probabilities. At step 764, in various embodiments, the system separates the nuclei groups into subgroups (e.g., a group of one or more nuclei within a group, grouped together based on physical proximity, shape, etc.). For example, the system, in one embodiment, iteratively splits the longest edge of the spanning tree into subgroups until a minimum length of the spanning tree is achieved (e.g., 10 microns).

In various embodiments, at step 766, the system calculates the features of each subgroup that are relevant to a determination of abnormality. For example, the system may calculate the width, length, area, and aspect ratio of the group's convex hull; width, length, area, and aspect ratio of the group's locally-convex hull (e.g., the hull that would be created if there was a maximum allowed edge length in the convex hull); number of nuclei in the group; inverse strength of the nuclei's abnormality probability (e.g., the negative of the log of the mean probability of the group); number of benign nuclei (e.g., nuclei whose abnormality probability is below the threshold to be considered for grouping) near or within the group's boundary; mean and median probability of benign nuclei near or within the group's boundary; other aggregations of individual nuclei features including size and shape variability measures, texture measures of the nuclear interiors, mean, median, or other order statistics of the probability of a nucleus being a histiocyte, and normalized color measures inside the nuclei; and other features of the areas between the nuclei but inside (or near) the group including texture measures of the extra-nuclear area, normalized color measures, and aggregate statistics of approximations to the nuclear/cytoplasm area ratio for the cells included in the group. At step 768, in various embodiments, the system classifies the subgroups based on the calculated features to generate a probability of abnormality for each group/subgroup (e.g., the likelihood that the group/subgroup comprises the abnormality). Thus, at step 770, in various embodiments, the system calculates a probability of abnormality for the region of interest (e.g., the likelihood that the region of interest comprises the abnormality). In one embodiment, the probability of abnormality for a region of interest is the maximum of the probability of abnormality for all of the groups/subgroups within the region of interest. Generally, at step 772, the system flags a region of interest for further review by a professional (e.g., pathologist) if the probability of abnormality for the region of interest is above a predetermined threshold. As will occur to one having ordinary skill in the art, the predetermined threshold is determined based on the number of regions of interest that the professional wishes to be flagged (e.g., a lower predetermined threshold will result in more flagged regions of interest). Thus, at step 774, the results of the exemplary regional analysis process 700E are stored with the histopathology image for subsequent analysis by the professional and the exemplary regional analysis process 700E ends thereafter. In one embodiment, the system automatically determines whether the histopathology images comprises the abnormality and does not pass the image along to a professional for confirmation of the same. To further understand the tissue analysis process, a description of exemplary histopathology images may be helpful.

Referring now to FIG. 8 (consisting of FIGS. 8A, 8B, 8C, 8D, 8E, 8F, 8G, and 8H), exemplary histopathology images are shown according to one embodiment of the present disclosure. Generally, FIG. 8A illustrates an exemplary histopathology image 800A prior to processing according to the present disclosure (e.g., after being processed through slide scanner 106 from FIG. 1) with exemplary tissue 802 and non-tissue background 804 visible. For example, tissue 802 may correspond to an H&E stained tissue sample from a patient's lymph node, etc. In one embodiment, the exemplary histopathology image 800A may comprise only exemplary tissue 802 and may not have any background 804 visible.

In one embodiment, FIG. 8B illustrates an exemplary histopathology image 800B after step 614 in the exemplary low-resolution analysis process 600 (from FIG. 6) with exemplary selected potential regions of interest 806 (generated by the system) visible and a tissue mask applied to the non-tissue background 804 by the system so that it is excluded from the processing.

FIG. 8C, in one embodiment, illustrates an exemplary histopathology image 800C after the exemplary low-resolution analysis process 600 (from FIG. 6) with exemplary identified potential regions of interest 808 (generated by the system) visible. In various embodiments, the system may indicate identified potential regions of interest 808 in other manners (e.g., squares, shaded regions, etc.).

In one embodiment, FIG. 8D illustrates an exemplary, high-resolution histopathology image 800D after the exemplary low-resolution analysis process 600 (from FIG. 6) with exemplary identified potential regions of interest 808 (generated by the system) and annotation lines 810 visible (e.g., as would be drawn by a professional prior to analysis by the system).

FIG. 8E, in one embodiment, illustrates an exemplary histopathology image 800E after step 708 in the exemplary high-resolution analysis process 700 (from FIG. 7) with exemplary segmented and classified nuclei, both abnormal 812 and benign 814, visible, as would be generated by the system. Generally, the system may indicate the segmented and classified abnormal 812 and benign 814 nuclei in other manners (e.g., two different/distinct colors, two different/distinct shapes, etc.).

FIG. 8F, in one embodiment, illustrates an exemplary histopathology image 800F during the exemplary high-resolution analysis process 700 (from FIG. 7) with exemplary nuclei groups 816 and subgroups 818 visible, as would be generated by the system. Generally, the system may indicate the exemplary nuclei groups 816 and subgroups 818 in other manners (e.g., two different/distinct colors, two different/distinct shapes, two different/distinct types of shading, etc.).

In one embodiment, FIG. 8G illustrates an exemplary histopathology image 800G after the exemplary high-resolution analysis process 700 (from FIG. 7) with exemplary flagged regions of interest 820 visible, as would be generated by the system. In various embodiments, the system may indicate the exemplary flagged regions of interest 820 in other manners (e.g., square-shaped highlights, particular symbol, textured shading, etc.).

FIG. 8H, in one embodiment, illustrates an alternative exemplary histopathology image 800H after the exemplary high-resolution analysis process 700 (from FIG. 7) with exemplary flagged regions of interest 820 visible, as would be generated by the system. Generally, any of the annotations (e.g., exemplary flagged regions of interest 820, exemplary nuclei groups 816 and subgroups 818, exemplary segmented and classified nuclei 812 and 814, etc.) may be toggled on/off by the professional while viewing the histopathology image as the annotations are stored as layers with the histopathology image.

From the foregoing, it will be understood that various aspects of the processes described herein are software processes that execute on computer systems that form parts of the system. Accordingly, it will be understood that various embodiments of the system described herein are generally implemented as specially-configured computers including various computer hardware components and, in many cases, significant additional features as compared to conventional or known computers, processes, or the like, as discussed in greater detail herein. Embodiments within the scope of the present disclosure also include computer-readable media for carrying or having computer-executable instructions or data structures stored thereon. Such computer-readable media can be any available media which can be accessed by a computer, or downloadable through communication networks. By way of example, and not limitation, such computer-readable media can comprise various forms of data storage devices or media such as RAM, ROM, flash memory, EEPROM, CD-ROM, DVD, or other optical disk storage, magnetic disk storage, solid state drives (SSDs) or other data storage devices, any type of removable non-volatile memories such as secure digital (SD), flash memory, memory stick, etc., or any other medium which can be used to carry or store computer program code in the form of computer-executable instructions or data structures and which can be accessed by a general purpose computer, special purpose computer, specially-configured computer, mobile device, etc.

When information is transferred or provided over a network or another communications connection (either hardwired, wireless, or a combination of hardwired or wireless) to a computer, the computer properly views the connection as a computer-readable medium. Thus, any such a connection is properly termed and considered a computer-readable medium. Combinations of the above should also be included within the scope of computer-readable media. Computer-executable instructions comprise, for example, instructions and data which cause a general purpose computer, special purpose computer, or special purpose processing device such as a mobile device processor to perform one specific function or a group of functions.

Those skilled in the art will understand the features and aspects of a suitable computing environment in which aspects of the disclosure may be implemented. Although not required, some of the embodiments of the claimed inventions may be described in the context of computer-executable instructions, such as program modules or engines, as described earlier, being executed by computers in networked environments. Such program modules are often reflected and illustrated by flow charts, sequence diagrams, exemplary screen displays, and other techniques used by those skilled in the art to communicate how to make and use such computer program modules. Generally, program modules include routines, programs, functions, objects, components, data structures, application programming interface (API) calls to other computers whether local or remote, etc. that perform particular tasks or implement particular defined data types, within the computer. Computer-executable instructions, associated data structures and/or schemas, and program modules represent examples of the program code for executing steps of the methods disclosed herein. The particular sequence of such executable instructions or associated data structures represent examples of corresponding acts for implementing the functions described in such steps.

Those skilled in the art will also appreciate that the claimed and/or described systems and methods may be practiced in network computing environments with many types of computer system configurations, including personal computers, smartphones, tablets, hand-held devices, multi-processor systems, microprocessor-based or programmable consumer electronics, networked PCs, minicomputers, mainframe computers, and the like. Embodiments of the claimed invention are practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing various aspects of the described operations, which is not illustrated, includes a computing device including a processing unit, a system memory, and a system bus that couples various system components including the system memory to the processing unit. The computer will typically include one or more data storage devices for reading data from and writing data to. The data storage devices provide nonvolatile storage of computer-executable instructions, data structures, program modules, and other data for the computer.

Computer program code that implements the functionality described herein typically comprises one or more program modules that may be stored on a data storage device. This program code, as is known to those skilled in the art, usually includes an operating system, one or more application programs, other program modules, and program data. A user may enter commands and information into the computer through keyboard, touch screen, pointing device, a script containing computer program code written in a scripting language or other input devices (not shown), such as a microphone, etc. These and other input devices are often connected to the processing unit through known electrical, optical, or wireless connections.

The computer that effects many aspects of the described processes will typically operate in a networked environment using logical connections to one or more remote computers or data sources, which are described further below. Remote computers may be another personal computer, a server, a router, a network PC, a peer device or other common network node, and typically include many or all of the elements described above relative to the main computer system in which the inventions are embodied. The logical connections between computers include a local area network (LAN), a wide area network (WAN), virtual networks (WAN or LAN), and wireless LANs (WLAN) that are presented here by way of example and not limitation. Such networking environments are commonplace in office-wide or enterprise-wide computer networks, intranets, and the Internet.

When used in a LAN or WLAN networking environment, a computer system implementing aspects of the invention is connected to the local network through a network interface or adapter. When used in a WAN or WLAN networking environment, the computer may include a modem, a wireless link, or other mechanisms for establishing communications over the wide area network, such as the Internet. In a networked environment, program modules depicted relative to the computer, or portions thereof, may be stored in a remote data storage device. It will be appreciated that the network connections described or shown are exemplary and other mechanisms of establishing communications over wide area networks or the Internet may be used.

While various aspects have been described in the context of a preferred embodiment, additional aspects, features, and methodologies of the claimed inventions will be readily discernible from the description herein, by those of ordinary skill in the art. Many embodiments and adaptations of the disclosure and claimed inventions other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed inventions. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed inventions. In addition, some steps may be carried out simultaneously, contemporaneously, or in synchronization with other steps.

The embodiments were chosen and described in order to explain the principles of the claimed inventions and their practical application so as to enable others skilled in the art to utilize the inventions and various embodiments and with various modifications as are suited to the particular use contemplated. Alternative embodiments will become apparent to those skilled in the art to which the claimed inventions pertain without departing from their spirit and scope. Accordingly, the scope of the claimed inventions is defined by the appended claims rather than the foregoing description and the exemplary embodiments described therein.

1. A method for processing images of cells to identify cellular nuclei within the cells for use in connection with identifying a possible abnormality with respect to the cells, comprising the steps of:
receiving an image of one or more cells, each cell having a cellular nucleus, wherein the image of the one or more cells comprises a plurality of pixels of varying brightness;
applying a sampling matrix to each of the plurality of pixels of the image of the one or more cells, wherein the sampling matrix determines one or more first and second derivatives with respect to the brightness of a particular pixel to which the sampling matrix was applied;
determining a consistency for each of the one or more first and second derivatives; and
selecting, based on the determined consistency for each of the one or more first and second derivatives, one or more edges of a cellular nucleus within the image of the one or more cells, wherein the selected one or more edges of the cellular nucleus help define the shape of the cellular nucleus.

2. The method of claim 1, wherein the sampling matrix comprises an arc-shaped filter.

3. (canceled)

4. (canceled)

5. (canceled)

6. The method of claim 1, wherein selecting the one or more edges of the cellular nucleus within the image of the one or more cells further comprises the steps of:
converting the determined consistency for each of the one or more first and second derivatives into a normalized signal-to-noise ratio value; and
selecting the one or more edges of the cellular nucleus within the image of the one or more cells corresponding to the determined consistency for each of the one or more first and second derivatives with the maximum normalized signal-to-noise ratio value.

7. The method of claim 1, wherein the image of the one or more cells comprises a preprocessed image of the one or more cells.

8. (canceled)

9. (canceled)

10. (canceled)

11. (canceled)

12. (canceled)

13. (canceled)

14. (canceled)

15. (canceled)

16. A method for processing images of cells to identify cellular nuclei within the cells and to determine nuclei shapes within the cells for use in connection with identifying a possible abnormality with respect to the cells, comprising the steps of:
receiving an image of one or more cells, each cell having a cellular nucleus, wherein the image of the one or more cells comprises a plurality of pixels of varying brightness and shape data regarding at least one particular nucleus within the image of the one or more cells;
selecting, based on the shape data, an initial pixel within the at least one particular nucleus from which to determine the shape of the at least one particular nucleus;
adding additional pixels to the initial pixel, based on one or more predefined rules, until the number of pixels within the at least one particular nucleus exceeds a predetermined threshold value; and
determining, based on the additional pixels, the shape of the at least one particular nucleus.

17. The method of claim 16, wherein the shape data comprises data corresponding to one or more edges of the at least one particular nucleus and data regarding one or more initial pixels within the at least particular one nucleus.

18. The method of claim 16, wherein the one or more predefined rules define, based on one or more multivariate normal distribution intensities of the brightness of the additional pixels, the additional pixels most likely to be within the at least particular one nucleus.

19. The method of claim 18, wherein the one or more multivariate normal distribution intensities are determined based on the brightness of the additional pixels and the shape data.

20. The method of claim 16, wherein the shape data comprises the predetermined threshold value.

21. The method of claim 16, further comprising the step of determining, after each additional pixel is added to the initial pixel, a fitness of a current shape of the at least one particular nucleus, wherein the fitness corresponds to the accuracy of the current shape of the at least one particular nucleus.

22. The method of claim 21, wherein the shape of the at least one particular nucleus is determined based on the fitness determined after each additional pixel was added to the initial pixel.

23. The method of claim 16, wherein the image of the one or more cells comprises a preprocessed image of the one or more cells.

24. (canceled)

25. (canceled)

26. (canceled)

27. (canceled)

28. (canceled)

29. (canceled)

30. (canceled)

31. (canceled)

32. (canceled)

33. A method for processing images of cells comprising cellular nuclei to determine the presence of an abnormality within the cells, comprising the steps of:
receiving an image of one or more cells, each cell having a cellular nucleus, wherein the image of the one or more cells comprises a plurality of pixels of varying brightness;
identifying, based on the brightness of the plurality of pixels, one or more edges corresponding to a particular cellular nucleus;
defining, based on the identified one or more edges and the plurality of pixels, a shape of the particular cellular nucleus; and
comparing the shape of the particular cellular nucleus to one or more predefined rules to determine whether the shape of the particular cellular nucleus indicates the presence of the abnormality in the one or more cells.

34. The method of claim 33, wherein identifying the one or more edges further comprises the steps of:
applying a sampling matrix to each of the plurality of pixels of the image of the one or more cells, wherein the sampling matrix determines one or more first and second derivatives with respect to the brightness of a particular pixel to which the sampling matrix was applied;
determining a consistency for each of the one or more first and second derivatives; and
selecting, based on the determined consistency for each of the one or more first and second derivatives, one or more edges of a cellular nucleus within the image of the one or more cells, wherein the selected one or more edges of the cellular nucleus help define the shape of the cellular nucleus.

35. The method of claim 33, wherein defining the shape of the particular cellular nucleus further comprises the steps of:
selecting, based on the identified one or more edges and the plurality of pixels, an initial pixel within the one particular nucleus from which to determine the shape of the particular nucleus;
adding additional pixels to the initial pixel, based on one or more predefined rules, until the number of pixels within the particular nucleus exceeds a predetermined threshold value; and
determining, based on the additional pixels, the shape of the particular nucleus.

36. The method of claim 33, wherein the one or more predefined rules comprise data regarding the characteristics of cellular nuclei comprising the particular abnormality.

37. The method of claim 36, wherein the characteristics of nuclei are selected from the group comprising: a shape of the cellular nuclei, a size of the cellular nuclei, a spatial relationship between the cellular nuclei, and a number of the cellular nuclei within a region of predetermined size.

38. The method of claim 33, further comprising the step of, prior to identifying the one or more edges, preprocessing the image of the one or more cells.

39. The method of claim 38, wherein preprocessing the image of the one or more cells further comprises the step of identifying tissue comprising the one or more cells within the image of the one or more cells.

40. The method of claim 38, wherein preprocessing the image of the one or more cells further comprises the steps of identifying one or more artifacts within the image of the one or more cells and removing the identified one or more artifacts from the image of the one or more cells.

41. The method of claim 38, wherein preprocessing the image of the one or more cells further comprises the step of converting the image of the one or more cells to a particular color space.

42. The method of claim 38, wherein preprocessing the image of the one or more cells further comprises the step of extracting one or more particular color channels from the image of the one or more cells.

43. The method of claim 38, wherein preprocessing the image of the one or more cells further comprises the step of selecting a particular image size for the image of the one or more cells.

44. The method of claim 38, wherein preprocessing the image of the one or more cells further comprises the step of identifying one or more texture features within the image of the one or more cells.

45. The method of claim 38, wherein preprocessing the image of the one or more cells further comprises the step of dividing the plurality of pixels into one or more groups of predetermined size.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)
